(19) United States
(12) Patent Application Publication
(10) Pub. No.: US 2026/0125734 A1
(43) Pub. Date: May 7, 2026

(54) SYSTEMS, DEVICES, AND METHODS FOR CONDUCTING ANTIMICROBIAL SUSCEPTIBILITY TESTING

(71) Applicant: Avails Medical, Inc., Menlo Park, CA (US)

(72) Inventors: Creighton T. BUIE, Menlo Park, CA (US); Ankit BAINGANE, Menlo Park, CA (US); Tatiana LAM, Menlo Park, CA (US); Elizabeth Botbol PONTE, Menlo Park, CA (US); Kristen Lugenie COTNER, Menlo Park, CA (US); Nitin K. RAJAN, Menlo Park, CA (US); Oren S. KNOPFMACHER, Menlo Park, CA (US); Meike HERGET, Menlo Park, CA (US); Suzanne PUTNEY, Menlo Park, CA (US); Andrew H. THEISS, Menlo Park, CA (US)

(73) Assignee: Avails Medical, Inc., Menlo Park, CA (US)

(21) Appl. No.: 19/374,666

(22) Filed: Oct. 30, 2025

Related U.S. Application Data
(60) Provisional application No. 63/715,378, filed on Nov. 1, 2024.

(51) Int. Cl. C12Q 1/18; G01N 27/327

(52) U.S. Cl. CPC C12Q 1/18; G01N 27/3277

(57) ABSTRACT

Various apparatus, systems, and methods for determining a susceptibility of a bacteria to an antibiotic are disclosed. In one aspect, a reader comprises at least one reader module configured to receive a well plate covered by a sensor array lid. Wells of the well plate contain aliquots of a sample comprising the bacteria. The wells also comprise test wells containing the antibiotic and at least one control well without the antibiotic. The reader module further comprises conductive connectors configured to contact conductive pads of the sensor array lid. One or more processors of the reader are programmed to determine the susceptibility of the bacteria to the antibiotic based on any changes in a solution characteristic of the aliquots within the test wells compared to the control well. The reader is also configured to perform certain internal quality checks when determining the susceptibility of the bacteria to the antibiotic.

### CROSS-REFERENCE TO RELATED APPLICATIONS

This application claims the benefit of and priority to U.S. Provisional Patent Application No. 63/715,378 filed on Nov. 1, 2024, the content of which is incorporated herein by reference in its entirety.

### STATEMENT AS TO FEDERALLY SPONSORED RESEARCH

This invention was made with U.S. Government support under Agreement Number 75A50122C00028, awarded by the U.S. Department of Health and Human Services. The U.S. Government has certain rights in the invention.

### TECHNICAL FIELD

The present disclosure relates generally to diagnostic testing and, more specifically, to systems, devices, and methods for conducting antimicrobial susceptibility testing.

### BACKGROUND

An increasing number of pathogenic bacteria are acquiring antibiotic resistance and new forms of resistance are continuously emerging with alarming speed across international boundaries. The U.S. Center for Disease Control (CDC) considers antimicrobial resistance as one of the biggest public health challenges of our time. Every year in the U.S. alone, over 2 million people acquire antibiotic-resistant infections and death rates are continuously rising. Providing a rapid and low-cost antibiotic susceptibility test (AST) will be crucial in controlling this burgeoning problem. Current gold standard AST tests still often require burdensome and time-consuming overnight culturing steps. This has prompted a push for rapid AST tests that can provide results in a matter of hours. Speeding up AST results to provide targeted antibiotic therapy early on is key to improving patient survival. Delays in obtaining AST results can lead to healthcare professionals having no choice but to administer broad-spectrum antibiotics, which can promote antibiotic resistance (AR). While new technologies are under development, most still require a culture isolate as an input.

Therefore, a solution is needed that can detect phenotypic bacterial growth in samples containing a patient's blood (e.g., a positive blood culture) that does not need to go through the bacterial isolation steps of traditional testing procedures. Such a solution should not be overly complex and should be cost-effective to manufacture. Such a solution should also not rely on labor-intensive techniques and provide accurate results. Such a solution should also allow for multiplex detection involving simultaneous readout of multiple wells to rapidly determine minimum inhibitory concentrations (MICs).

### SUMMARY

Disclosed herein are systems, devices, and methods for conducting antimicrobial susceptibility testing. In some embodiments, a reader for determining the susceptibility of a bacteria to an antibiotic comprises a plurality of reader modules. Each of the reader modules can comprise a moveable or slidable plate tray configured to receive a well plate covered by a sensor array lid. Each of the reader modules can also comprise a printed circuit board assembly (PCBA) disposed above the plate tray when the plate tray is inserted or otherwise retracted into the reader. The well plate can comprise a plurality of wells configured to contain aliquots of a sample including the bacteria. The wells of the well plate can comprise test wells containing the antibiotic and at least one control well devoid of any antibiotic. The PCBA can comprise a plurality of conductive connectors. The conductive connectors can extend downward from an underside of the PCBA to contact conductive pads of the sensor array lid. Sensor units of the sensor array lid can be configured to be immersed in the aliquots of the sample within the well plate. The reader can further comprise one or more processors and one more memory units. The one or more processors can be programmed to determine the susceptibility of the bacteria to the antibiotic based on any changes in a solution characteristic of the aliquots within the test wells compared to the control well over time.

In some embodiments, the conductive connectors can be leaf spring connectors. The leaf spring connectors can be made in part of a conductive metal or metal alloy.

In some embodiments, each of the reader modules can further comprise a gasket disposed in between the PCBA and the sensor array lid covering the well plate placed on the plate tray. The gasket can be configured to create a partial seal around a top of the sensor array lid to control an evaporation rate of the aliquots of the sample and to control a humidity level and a partial pressure of oxygen within a test headspace above the well plate.

In some embodiments, the gasket can be made in part of a semipermeable polymeric material having a Shore A hardness between about 20 to 55. In certain embodiments, the gasket can be made in part of a semipermeable polymeric material having a Shore A hardness of between about 45 to 50. The gasket can act as a perimeter surrounding the portions of the conductive connectors extending downward from the underside of the PCBA.

In some embodiments, each of the reader modules can further comprise an upper heater coupled to or integrated with the PCBA. The upper heater can be configured to control condensation on the conductive connectors by heating the conductive connectors above a dew point.

In some embodiments, each of the reader modules can further comprise a lower heater coupled to the plate tray. The well plate can be configured to be placed above the lower heater. The lower heater can be configured to heat the well plate to ensure optimal bacterial growth conditions within the wells of the well plate.

In some embodiments, each of the reader modules can further comprise a first temperature sensor configured to monitor the temperature of the lower heater used to heat the well plate.

In some embodiments, each of the reader modules can further comprise a second temperature sensor and a humidity sensor to monitor the temperature and the humidity, respectively, within a test headspace above the well plate.

In some embodiments, the sensor units of the sensor array lid can comprise oxidation-reduction potential (ORP) sensors. In these embodiments, the one or more processors can be further programmed to perform an internal quality check during each run. The internal quality check can comprise checking that a baseline voltage of a starting ORP of each of the aliquots of the sample within each of the wells is between about 2600 millivolts (mV) and 2750 mV (e.g., between about 100 mV and 250 mV, plus the 2.5V DC offset).

Also, in embodiments where the sensor units comprise ORP sensors, the one or more processors can be further programmed to perform an internal quality check during each run. The internal quality check can comprise checking that a voltage noise of each of the ORP sensors is between about 0 millivolt root-mean-square (mVrms) and 5 mVrms.

Moreover, in embodiments where the sensor units comprise ORP sensors, the one or more processors can be further programmed to perform an internal quality check during each run. The internal quality check can comprise checking that a sensor voltage drift of each of the ORP sensors is between about 0 mV per hour and 40 mV per hour.

In some embodiments, the reader can further comprise a reader housing and each of the reader modules can further comprise a tray carrier configured to automatically translate or drive the plate tray at least partially out of the reader housing to receive the well plate covered by the sensor array lid. In these embodiments, the conductive connectors can be aligned to contact the conductive pads of the sensor array lid after the plate tray loaded with the well plate covered by the sensor array lid is automatically retracted back into the reader housing by the tray carrier.

In some embodiments, the sensor array lid can further comprise a lid cover and a sensor substrate layer coupled to an underside of the lid cover. The lid cover can comprise a plurality of openings configured to expose the conductive pads. The conductive connectors can be configured to extend into the openings to contact the conductive pads.

Also disclosed is another embodiment of a reader for determining the susceptibility of a bacteria to an antibiotic. The reader can comprise a plurality of reader modules. Each of the reader modules can comprise a plate tray configured to receive a well plate covered by a sensor array lid. The well plate can comprise a plurality of wells configured to contain aliquots of a sample including the bacteria. The wells of the well plate can comprise test wells containing the antibiotic and at least one control well devoid of any antibiotic. Each of the reader modules can comprise a printed circuit board assembly (PCBA) disposed above the plate tray and configured to be in electrical contact with the sensor array lid. The sensor array lid can comprise a plurality of sensor units configured to be immersed in the aliquots of the sample within the well plate. Each of the reader modules can also comprise a gasket disposed in between the PCBA and the sensor array lid. The gasket can be configured to create a partial seal around a top of the sensor array lid to control an evaporation rate of the aliquots of the sample and a humidity level and a partial pressure of oxygen within a space above the well plate. The reader can further comprise one or more processors and one more memory units. The one or more processors can be programmed to determine the susceptibility of the bacteria to the antibiotic based on any changes in a solution characteristic of the aliquots within the test wells compared to the control well over time.

In some embodiments, the gasket can be made in part of a semipermeable polymeric material. For example, the gasket can be made of silicone rubber.

In certain embodiments, the gasket can be made of a material having a Shore A hardness of between about 20 to 55. In certain embodiments, the gasket can be made of a material having a Shore A hardness of between about 45 to 50. The gasket can act as a perimeter surrounding the portions of the conductive connectors extending downward from the underside of the PCBA.

In some embodiments, the space above the well plate can be created in part by the gasket and the sensor array lid.

In some embodiments, each of the reader modules can further comprise a gasket plate. The gasket plate can be disposed in between the PCBA and the sensor array lid. The gasket can be coupled to an underside of the gasket plate.

In some embodiments, the gasket can be substantially shaped as a polygon. In other embodiments, the gasket can be substantially shaped as an oval or a circle.

In some embodiments, the PCBA can comprise a plurality of conductive connectors extending downward from an underside of the PCBA to electrically contact conductive pads of the sensor array lid. The gasket can serve as a perimeter surrounding the portions of the conductive connectors extending downward from the underside of the PCBA.

In some embodiments, each of the reader modules can further comprise an upper heater coupled to or integrated with the PCBA. The upper heater can be configured to control condensation on the conductive connectors by heating the conductive connectors above a dew point.

In some embodiments, the reader can comprise a reader housing and each of the reader modules can further comprise a tray carrier configured to automatically translate or drive the plate tray at least partially out of the reader housing to receive the well plate covered by the sensor array lid. In these embodiments, the conductive connectors can be aligned to electrically contact the conductive pads of the sensor array lid after the plate tray loaded with the well plate covered by the sensor array lid is automatically retracted back into the reader housing by the tray carrier.

In some embodiments, each of the reader modules can further comprise a temperature sensor and a humidity sensor to monitor a temperature and a humidity level, respectively, within the space above the well plate.

In some embodiments, each of the reader modules can further comprise a lower heater coupled to the plate tray. The well plate can be configured to be placed above the lower heater. The lower heater can be configured to heat the well plate to ensure optimal bacterial growth conditions.

In some embodiments, the sensor units of the sensor array lid can comprise oxidation-reduction potential (ORP) sensors. In these embodiments, the one or more processors of the reader can be further programmed to perform an internal quality check during each run. The internal quality check can comprise: (i) checking that a baseline voltage of a starting ORP of each of the aliquots of the sample within the wells is between about 2600 millivolts (mV) and 2750 mV (e.g., between about 100 mV and 250 mV, plus the 2.5V DC offset); (ii) checking that a voltage noise of each of the ORP sensors is between about 0 millivolt root-mean-square (mVrms) and 5 mVrms; and (iii) checking that a sensor voltage drift of each of the ORP sensors is between about 0 mV per hour and 40 mV per hour.

Further disclosed is yet another embodiment of a reader for determining a susceptibility of a bacteria to an antibiotic. The reader can comprise a plurality of reader modules. Each of the reader modules can comprise a plate tray configured to receive a well plate covered by a sensor array lid. The well plate can comprise a plurality of wells configured to contain aliquots of a sample including the bacteria. The wells can comprise test wells containing the antibiotic and at least one control well devoid of any antibiotic. The sensor array lid can comprise a plurality of oxidation-reduction potential (ORP) sensors. Each of the reader modules can further comprise a printed circuit board assembly (PCBA) disposed above the plate tray and configured to be in electrical contact with the sensor array lid. Parts of the ORP sensors can be configured to be immersed in the aliquots of the sample within the well plate. The reader can also comprise one or more processors and one more memory units. The one or more processors can be programmed to check that a baseline voltage of a starting ORP of each of the aliquots of the sample within the wells is between about 2600 millivolts (mV) and 2750 mV (e.g., between about 100 mV and 250 mV, plus the 2.5V DC offset), a voltage noise of each of the ORP sensors is between about 0 millivolt root-mean-square (mVrms) and 5 mVrms, and a sensor voltage drift of each of the ORP sensors is between about 0 mV per hour and 40 mV per hour, and determine the susceptibility of the bacteria to the antibiotic based on any changes in a solution characteristic of the aliquots within the test wells compared to the control well over time.

In some embodiments, the reader can further comprise a gasket disposed in between the PCBA and the sensor array lid. The gasket can be configured to create a partial seal around a top of the sensor array lid to control an evaporation rate of the aliquots of the sample and a humidity level and a partial pressure of oxygen within a space above the well plate. The gasket can be made in part of a semipermeable polymeric material having a Shore A hardness of between about 20 to 55 (e.g., between about 40 and 50).

In some embodiments, the space above the well plate can be created in part by the gasket and the sensor array lid.

In some embodiments, the reader can further comprise a gasket plate. The gasket plate can be disposed in between the PCBA and the sensor array lid. The gasket can be coupled to an underside of the gasket plate.

In some embodiments, each of the reader modules can further comprise a temperature sensor and a humidity sensor to monitor a temperature and a humidity level, respectively, within a space above the well plate.

In some embodiments, the PCBA can further comprise a plurality of conductive connectors extending downward from an underside of the PCBA to electrically contact conductive pads of the sensor array lid.

In some embodiments, each of the reader modules can further comprise an upper heater coupled to or integrated with the PCBA, and the upper heater can be configured to control condensation on the conductive connectors by heating the conductive connectors above a dew point.

In some embodiments, the reader can further comprise a reader housing and each of the reader modules can further comprise a tray carrier configured to automatically translate or drive the plate tray at least partially out of the reader housing to receive the well plate covered by the sensor array lid. The conductive connectors can be aligned to electrically contact the conductive pads of the sensor array lid after the plate tray loaded with the well plate covered by the sensor array lid is automatically retracted back into the reader housing by the tray carrier.

In some embodiments, each of the reader modules can further comprise a lower heater coupled to the plate tray. The well plate can be configured to be placed above the lower heater. The lower heater can be configured to heat the well plate to ensure bacterial growth.

Also disclosed are one or more non-transitory computer-readable media comprising instructions stored thereon, that when executed by one or more processors, cause the one or more processors to perform operations including: checking that a baseline voltage of a starting oxidation-reduction potential (ORP) of each aliquot of a sample within wells of a well plate is between about 2600 millivolts (mV) and 2750 mV, checking that a voltage noise of each of the ORP sensors is between about 0 millivolt root-mean-square (mVrms) and 5 mVrms, and checking that a sensor voltage drift of each of the ORP sensors is between about 0 mV per hour and 40 mV per hour. The sample can comprise bacteria and the wells of the well plate can comprise test wells containing an antibiotic and at least one control well devoid of any antibiotic. The well plate can be covered by a sensor array lid comprising a plurality of ORP sensors configured to be immersed in the aliquots of the sample within the well plate. The well plate can be placed on a plate tray of a reader module and the reader module can further comprise a printed circuit board assembly (PCBA) disposed above the plate tray and configured to be in electrical contact with the sensor array lid. The one or more non-transitory computer-readable media can further comprise instructions stored thereon, that when executed by one or more processors, cause the one or more processors to determine the susceptibility of the bacteria to the antibiotic based on any changes in a solution characteristic of the aliquots within the test wells compared to the control well over time.

In some embodiments, a method of determining the susceptibility of a bacteria to an antibiotic can comprise placing a well plate covered by a sensor array lid on a plate tray of a reader module of a reader. The well plate can comprise a plurality of wells configured to contain aliquots of a sample comprising the bacteria. The wells of the well plate can comprise test wells containing the antibiotic and at least one control well devoid of any antibiotic. The sensor array lid can comprise sensor units configured to be immersed in the aliquots of the sample within the well plate. The method can also comprise pushing the plate tray into the reader or causing the plate tray to be retracted into the reader. A plurality of conductive connectors of a printed circuit board assembly (PCBA) disposed above the plate tray can be placed in electrical contact with conductive pads of the sensor array lid after the plate tray is fully pushed or retracted into the reader. The conductive connectors can extend downward from an underside of the PCBA to contact the conductive pads of the sensor array lid. The method can further comprise determining, using one or more processors, the susceptibility of the bacteria to the antibiotic based on any changes in a solution characteristic of the aliquots within the test wells compared to the control well over time.

In some embodiments, the conductive connectors can be leaf spring connectors.

In some embodiments, the reader module can further comprise a gasket. The gasket can be configured to create a partial seal around a top of the sensor array lid and the bottom of the PCBA to control an evaporation rate of the aliquots of the sample and a humidity level and a partial pressure of oxygen within a space above the well plate.

In some embodiments, the method can also comprise heating the conductive connectors above a dew point using an upper heater coupled to or integrated with the PCBA to control condensation on the conductive connectors.

In some embodiments, the method can also comprise monitoring a temperature and a humidity level within a space above the well plate using a temperature sensor and a humidity sensor.

In some embodiments, the method can also comprise heating the well plate using a lower heater coupled to the plate tray to ensure optimal bacterial growth conditions.

In some embodiments, the method can further comprise checking, using the one or more processors, that a baseline voltage of a starting ORP of each of the aliquots of the sample within the wells is between about 2600 millivolts (mV) and 2750 mV as part of an internal quality check (e.g., between about 100 mV and 250 mV, plus the 2.5V DC offset). In these embodiments, the sensor units can comprise ORP sensors.

In some embodiments, the method can also comprise checking, using the one or more processors, that a voltage noise of each of the sensor units is between about 0 millivolt root-mean-square (mVrms) and 5 mVrms as part of an internal quality check. In these embodiments, the sensor units can comprise ORP sensors.

In some embodiments, the method can further comprise checking, using the one or more processors, that a sensor voltage drift of each of the sensor units is between about 0 mV per hour and 40 mV per hour as part of an internal quality check. In these embodiments, the sensor units can comprise ORP sensors.

### BRIEF DESCRIPTION OF THE DRAWINGS

FIG. 1 illustrates one embodiment of a method of determining a susceptibility of a bacteria to an antibiotic and devices and systems used as part of the method.

FIG. 2 illustrates components of a sensor array lid of a testing device.

FIG. 3 illustrates a flexible substrate comprising a plurality of screen-printed sensors making up part of the sensor array lid and an underside of the sensor array lid with posts extending from the underside of a lid cover to bend the screen-printed sensors into position.

FIG. 4A is an exploded view illustrating components of a reader module of a reader.

FIG. 4B is another exploded view illustrating the components of the reader module.

FIG. 5A illustrates an underside of a printed circuit board assembly configured to engage with a testing device within the reader module.

FIG. 5B illustrates a bottom portion of the reader module with a testing device placed on a plate tray of the reader module.

FIG. 6 illustrates a side view of a reader module and a close-up view showing a gasket disposed in between a sensor array lid of a testing device and a printed circuit board assembly.

FIG. 7 is a side view illustrating a reader module with a testing device loaded on the reader module and the gasket removed for ease of viewing.

FIG. 8A is a graph illustrating ORP bacterial growth curves for various bacteria recorded using the system disclosed herein.

FIG. 8B is a graph illustrating an ORP signal obtained from a control well of the testing device and ORP signals obtained from test wells containing different concentrations of an antibiotic.

FIG. 9 is a graph illustrating ORP signals obtained from control wells of the testing device and ORP signals obtained from test wells containing different concentrations of the antibiotic tobramycin.

FIG. 10 is a graph illustrating ORP signals obtained from control wells of the testing device and ORP signals obtained from test wells containing different concentrations of the antibiotic piperacillin tazobactam.

FIG. 11 is a graph illustrating ORP signals obtained from control wells of the testing device and ORP signals obtained from test wells containing different concentrations of the antibiotic meropenem.

FIG. 12 is a graph illustrating ORP signals obtained from control wells of the testing device and ORP signals obtained from test wells containing different concentrations of the antibiotic ceftriaxone.

FIG. 13 is a graph illustrating ORP signals obtained from control wells of the testing device and ORP signals obtained from test wells containing different concentrations of the antibiotic ciprofloxacin.

FIG. 14 is a graph illustrating ORP signals obtained from control wells of the testing device and ORP signals obtained from test wells containing different concentrations of the antibiotic trimethoprim sulfamethoxazole.

FIG. 15 is a graph illustrating ORP signals obtained from control wells of the testing device and ORP signals obtained from test wells containing different concentrations of the antibiotic aztreonam.

### DETAILED DESCRIPTION

Variations of the devices, systems, and methods described herein are best understood from the detailed description when read in conjunction with the accompanying drawings. It is emphasized that, according to common practice, the various features of the drawings may not be to scale. The dimensions of certain features have been expanded or reduced for clarity and not all features may be visible or labeled in every drawing. The drawings are taken for illustrative purposes only and are not intended to define or limit the scope of the claims to that which is shown.

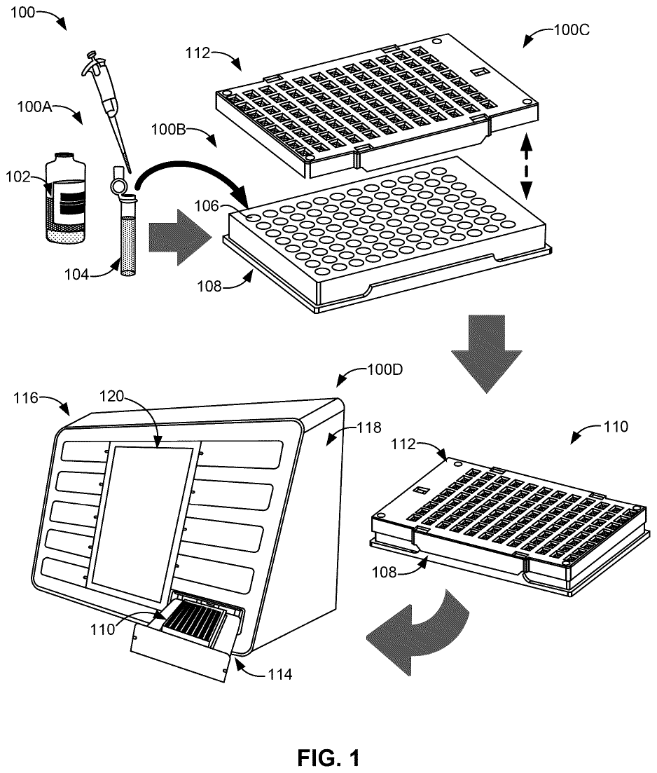

FIG. 1 illustrates example steps of an in vitro method 100 for determining a susceptibility of a bacteria to an antibiotic and certain devices used as part of the method 100. The method 100 can comprise introducing (e.g., via a micropipette or another type of fluid delivery device) an aliquot of a sample 102 comprising a bacteria into a reagent 104 contained within a reagent container (e.g., a test tube or reaction tube) to yield a standardized inoculum in step 100A.

In some embodiments, the sample 102 can comprise or refer to a bacterial culture derived from blood or another bodily fluid obtained from a patient or subject that has tested positive for microbial growth. When the sample 102 is a bacterial culture derived from blood, the sample 102 can be or be referred to as a positive blood culture (PBC).

For example, a patient can show symptoms of sepsis (e.g., high fever, chills, etc.). Blood (e.g., about 5 mL to about 10 mL) can be drawn from this patient and transferred to a commercial blood culturing container or vessel that contain bacterial growth media (e.g., 30 mL to 40 mL of growth media). The blood culturing container or vessel can then be incubated at 35° C.±2° C. to allow the bacteria to proliferate. If the patient's blood is contaminated with bacteria, the bacteria will replicate within the container/vessel and a blood culturing system or apparatus can determine the sample 102 as testing “positive” for bacterial growth. Such a PBC can then be used as the sample 102 for the method 100 disclosed herein.

In other embodiments, the sample 102 can comprise any combination of blood, urine, serum, plasma, saliva, sputum, semen, breast milk, joint fluid, spinal fluid such as cerebrospinal fluid, wound discharge, mucus, fluid accompanying stool, vaginal secretions, synovial fluid, pleural fluid, peritoneal fluid, pericardial fluid, and/or amniotic fluid.

In some embodiments, the sample 102 can comprise or refer to a bacterial culture derived from at least one of an environmental sample, a food sample, another type of biological sample, or a subject or patient. For example, the sample 102 can comprise or refer to a bacterial culture or a re-suspended bacterial culture derived from a bodily fluid or swab obtained from a subject or patient.

In certain embodiments, the subject or patient can be a human subject or patient. In other embodiments, the subject or patient can be a non-human animal subject or patient.

In some embodiments, the sample 102 can be confirmed to contain bacteria by a gram stain and then the bacteria can be identified using a rapid organism identification method (e.g., an FDA-cleared or laboratory-validated rapid organism ID method).

The aliquot of the sample 102 can be diluted when introduced into the reagent 104. For example, the aliquot of the sample 102 can be diluted by the reagent 104 by a dilution ratio of between about 1:10 and about 1:10000. As a more specific example, the aliquot of the sample 102 can be diluted by the reagent 104 by a dilution ratio of about 1:10, 1:50, 1:100, 1:200, 1:300, 1:400, 1:500, 1:600, 1:700, 1:800, 1:900, 1:1000, 1:2000, 1:3000, 1:4000, 1:5000, 1:6000, 1:7000, 1:8000, 1:9000, 1:10000, or any values therebetween.

The reagent 104 can be a growth inducer or nutrient solution. In some embodiments, the reagent 104 can be a Mueller Hinton (MH) broth such as a cation-adjusted Mueller Hinton Broth (CAMHB). In these embodiments, the reagent 104 can be a CAMHB combined with Pluronic®.

In other embodiments, the reagent 104 can comprise bacto-tryptone, yeast extract, beef extract, starch, glucose, acid hydrolysate of casein, calcium chloride, magnesium chloride, sodium chloride, a carbon-based inducer, a nitrogen-based inducer, a mineral, a trace element, a biological growth factor, blood or lysed blood including lysed horse blood (LHB), or a combination thereof (with or without CAMHB).

In some embodiments, the bacteria that can be assayed for antibiotic susceptibility using the method 100, system, and devices disclosed herein can comprise bacteria selected from the genera Acinetobacter, Acetobacter, Actinomyces, Aerococcus, Aeromonas, Agrobacterium, Anaplasma, Azorhizobium, Azotobacter, Bacillus, Bacteroides, Bartonella, Bordetella, Borrelia, Brucella, Burkholderia, Calymmatobacterium, Campylobacter, Chlamydia, Chlamydophila, Citrobacter, Clostridium, Corynebacterium, Coxiella, Ehrlichia, Enterobacter, Enterococcus, Escherichia, Francisella, Fusobacterium, Gardnerella, Haemophilus, Helicobacter, Klebsiella, Lactobacillus, Legionella, Listeria, Methanobacterium, Microbacterium, Micrococcus, Morganella, Moraxella, Mycobacterium, Mycoplasma, Neisseria, Pandoraea, Pasteurella, Peptostreptococcus, Porphyromonas, Prevotella, Proteus, Providencia, Pseudomonas, Ralstonia, Raoultella, Rhizobium, Rickettsia, Rochalimaea, Rothia, Salmonella, Serratia, Shewanella, Shigella, Spirillum, Staphylococcus, Strenotrophomonas, Streptococcus, Streptomyces, Treponema, Vibrio, Wolbachia, or Yersinia.

In certain embodiments, the bacteria that can be assayed for antibiotic susceptibility using the method 100, system, and devices disclosed herein can comprise Staphylococcus aureus, Staphylococcus lugdunensis, coagulase-negative Staphylococcus species (including but not limited to Staphylococcus epidermidis, Staphylococcus haemolyticus, Staphylococcus hominis, Staphylococcus capitis, not differentiated), Enterococcus faecalis, Enterococcus faecium (including but not limited to Enterococcus faecium and other Enterococcus spp., not differentiated, excluding Enterococcus faecalis), Streptococcus pneumoniae, Streptococcus pyogenes, Streptococcus agalactiae, Streptococcus spp., (including but not limited to Streptococcus mitis, Streptococcus pyogenes, Streptococcus gallolyticus, Streptococcus agalactiae, Streptococcus pneumoniae, not differentiated), Pseudomonas aeruginosa, Acinetobacter baumannii, Klebsiella spp. (including but not limited to Klebsiella pneumoniae, Klebsiella oxytoca, not differentiated), Escherichia coli, Enterobacter spp. (including but not limited to Enterobacter cloacae, Enterobacter aerogenes, not differentiated), Proteus spp. (including but not limited to Proteus mirabilis, Proteus vulgaris, not differentiated), Citrobacter spp. (including but not limited to Citrobacter freundii, Citrobacter koseri, not differentiated), or Serratia marcescens.

In additional embodiments, the bacteria that can be assayed for antibiotic susceptibility using the method 100, system, and devices disclosed herein can comprise Acinetobacter baumannii complex, Acinetobacter spp., Actinobacillus spp., Actinomycetes, Actinomyces spp. (including but not limited to Actinomyces israelii and Actinomyces naeslundii), Aeromonas spp. (including but not limited to Aeromonas hydrophila, Aeromonas veronii biovar sobria (Aeromonas sobria), and Aeromonas caviae), Anaplasma phagocytophilum, Alcaligenes xylosoxidans, Actinobacillus actinomycetemcomitans, Bacillus spp. (including but not limited to Bacillus anthracis, Bacillus cereus, Bacillus subtilis, Bacillus thuringiensis, and Bacillus stearothermophilus), Bacteroides spp. (including but not limited to Bacteroides fragilis), Bartonella spp. (including but not limited to Bartonella bacilliformis and Bartonella henselae, Bifidobacterium spp., Bordetella spp. (including but not limited to Bordetella pertussis, Bordetella parapertussis, and Bordetella bronchiseptica), Borrelia spp. (including but not limited to Borrelia recurrentis, and Borrelia burgdorferi), Brucella sp. (including but not limited to Brucella abortus, Brucella canis, Brucella melintensis and Brucella suis), Burkholderia spp. (including but not limited to Burkholderia pseudomallei and Burkholderia cepacia), Campylobacter spp. (including but not limited to Campylobacter jejuni, Campylobacter coli, Campylobacter lari and Campylobacter fetus), Capnocytophaga spp., Cardiobacterium hominis, Chlamydia trachomatis, Chlamydophila pneumoniae, Chlamydophila psittaci, Citrobacter spp. (including but not limited to Citrobacter freundii complex and Citrobacter koseri), Coxiella burnetii, Corynebacterium spp. (including but not limited to, Corynebacterium diphtheriae, Corynebacterium jeikeum and Corynebacterium), Clostridium spp. (including but not limited to Clostridium perfringens, Clostridium difficile, Clostridium botulinum and Clostridium tetani), Eikenella corrodens, Enterobacter spp. (including but not limited to Enterobacter aerogenes, Enterobacter agglomerans and Enterobacter cloacae complex), Escherichia coli, (including but not limited to enterotoxigenic E. coli, enteroinvasive E. coli, enteropathogenic E. coli, enterohemorrhagic E. coli, enteroaggregative E. coli and uropathogenic E. coli) Enterococcus spp. (including but not limited to Enterococcus faecalis and Enterococcus faecium) Ehrlichia spp. (including but not limited to Ehrlichia chafeensia and Ehrlichia canis), Erysipelothrix rhusiopathiae, Eubacterium spp., Francisella tularensis, Fusobacterium nucleatum, Gardnerella vaginalis, Gemella morbillorum, Haemophilus spp. (including but not limited to Haemophilus influenzae, Haemophilus ducreyi, Haemophilus aegyptius, Haemophilus parainfluenzae, Haemophilus haemolyticus and Haemophilus parahaemolyticus), Helicobacter spp. (including but not limited to Helicobacter pylori, Helicobacter cinaedi and Helicobacter fennelliae), Kingella kingii, Klebsiella spp. (including but not limited to Klebsiella pneumoniae, Klebsiella granulomatis and Klebsiella oxytoca), Lactobacillus spp., Listeria monocytogenes, Leptospira interrogans, Legionella pneumophila, Leptospira interrogans, Peptostreptococcus spp., Moraxella catarrhalis, Morganella spp. (including but not limited to Morganella morganii), Mobiluncus spp., Micrococcus spp., Mycobacterium spp. (including but not limited to Mycobacterium leprae, Mycobacterium tuberculosis, Mycobacterium intracellulare, Mycobacterium avium, Mycobacterium bovis, and Mycobacterium marinum), Mycoplasm spp. (including but not limited to Mycoplasma pneumoniae, Mycoplasma hominis, and Mycoplasma genitalium), Nocardia spp. (including but not limited to Nocardia asteroides, Nocardia cyriacigeorgica and Nocardia brasiliensis), Neisseria spp. (including but not limited to Neisseria gonorrhoeae and Neisseria meningitidis), Pasteurella multocida, Plesiomonas shigelloides. Prevotella spp., Porphyromonas spp., Prevotella melaninogenica, Proteus spp. (including but not limited to Proteus vulgaris and Proteus mirabilis), Providencia spp. (including but not limited to Providencia alcalifaciens, Providencia rettgeri and Providencia stuartii), Pseudomonas aeruginosa, Propionibacterium acnes, Rhodococcus equi, Rickettsia spp. (including but not limited to Rickettsia rickettsii, Rickettsia akari and Rickettsia prowazekii, Orientia tsutsugamushi (formerly: Rickettsia tsutsugamushi) and Rickettsia typhi), Rhodococcus spp., Stenotrophomonas maltophilia, Salmonella spp. (including but not limited to Salmonella enterica, Salmonella typhi, Salmonella paratyphi, Salmonella enteritidis, Salmonella cholerasuis and Salmonella typhimurium), Serratia spp. (including but not limited to Serratia marcescens and Serratia liquifaciens), Shigella spp. (including but not limited to Shigella dysenteriae, Shigella flexneri, Shigella boydii and Shigella sonnei), Staphylococcus spp. (including but not limited to Staphylococcus aureus, Staphylococcus epidermidis, Staphylococcus hemolyticus, Staphylococcus saprophyticus, Staphylococcus hominis, Staphylococcus warneri, Staphylococcus lugdunensis), Streptococcus spp. (including but not limited to Streptococcus pneumoniae, Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema spp. (including but not limited to Treponema carateum, Treponema petenue, Treponema pallidum and Treponema endemicum, Tropheryma whippelii, Ureaplasma urealyticum, Veillonella sp., Vibrio spp. (including but not limited to Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Xanthomonas maltophilia, or Yersinia spp. (including but not limited to Yersinia enterocolitica, Yersinia pestis, or Yersinia pseudotuberculosis).

The method 100, system, and devices disclosed herein can be used to assay both gram-negative and gram-positive bacteria for antibiotic susceptibility.

For example, Table 1 below lists certain gram-negative and gram-positive bacteria that can be assayed for antibiotic susceptibility using the method 100, system, and devices disclosed herein:

TABLE 1
Gram-negative and gram-positive bacteria that
can be assayed for antibiotic susceptibility

| Gram-negative bacteria | Gram-positive bacteria |
|---|---|
| Acinetobacter baumannii complex | Staphylococcus aureus |
| Citrobacter freundii complex | Staphylococcus lugdunensis |
| Citrobacter koseri | Staphylococcus epidermidis |
| Enterobacter aerogenes | Enterococcus faecalis |
| Enterobacter cloacae complex | Enterococcus faecium |
| Escherichia coli | |
| Klebsiella oxytoca | |
| Klebsiella pneumoniae | |
| Proteus mirabilis | |
| Proteus vulgaris | |
| Pseudomonas aeruginosa | |
| Serratia marcescens | |

Also, for example, Table 2 below lists dilution schemes for several example bacteria.

TABLE 2
Bacteria-specific Blood Culture Dilution Schemes

| | Dilution Volumes | % of positive blood |
|---|---|---|
| | (all volumes indicated | culture in reagent |
| Bacteria in positive | below are introduced | (e.g., CAMHB |
| blood culture | to 23.0 mL of reagent) | and Pluronic) |
| Escherichia coli | 15.0 μL | 0.07% |
| Klebsiella pneumoniae | | |
| Klebsiella oxytoca | | |
| Klebsiella aerogenes | | |
| Enterobacter cloacae complex | | |
| Citrobacter freundii complex | | |
| Citrobacter koseri | | |
| Serratia marcescens | | |
| Proteus mirabilis | | |
| Proteus vulgaris | | |
| Pseudomonas aeruginosa | 30.0 μL | 0.13% |
| Acinetobacter baumannii complex | 45.0 μL | 0.20% |

The method 100 can also comprise introducing (e.g., via a micropipette or another type of fluid delivery device) an aliquot of the diluted sample 102 (diluted in the reagent 104) comprising the bacteria into wells 106 of a well plate 108 in step 100B. The well plate 108 can be part of a testing device 110.

The testing device 110 can further comprise a sensor array lid 112. The sensor array lid 112 can be configured to cover or cap the well plate 108 when placed on top of the well plate 108.

The well plate 108 can comprise a plurality of wells 106 or microwells (the well plate 108 can also be referred to as a microwell plate). In some embodiments, the well plate 108 can comprise between 12 wells and 192 wells. For example, the well plate 108 can comprise 96 wells, 64 wells, or 48 wells. When the well plate 108 comprises 96 wells, the wells 106 of the well plate 108 can be arranged as an 8×12 array of wells. When the well plate 108 comprises 48 wells, the wells 106 of the well plate 108 can be arranged as a 6×8 array of wells.

In some embodiments, the wells 106 can be substantially bowl-shaped or hemispherical-shaped. The wells 106 can also be shaped as divots, depressions, or cavities. In other embodiments, the wells 106 can be shaped substantially as cylinders, upside-down conics, frustoconics or upside-down frustoconics, or partial ovoids. In additional embodiments, the wells 106 can be substantially cuboid-shaped.

In some embodiments, the well plate 108 can be made in part of a polymeric material or a thermoplastic. For example, the well plate 108 can be made in part of at least one of polystyrene, polypropylene, a cyclic olefin copolymer, or another biocompatible polymeric material.

In certain embodiments, the well plate 108 can be a commercially-available or off-the-shelf well plate such as a microtiter or microwell plate distributed by ThermoFisher Scientific, Beckman Coulter, VWR International, or MilliporeSigma. As a more specific example, the well plate 108 can be a commercially-available or off-the-shelf AST well plate.

In some embodiments, the wells 106 of the well plate 108 can comprise test wells and one or more control wells. The test wells can each comprise a type of antibiotic and the one or more control wells can be devoid of any antibiotic (e.g., serve as positive control wells).

In certain embodiments, the well plate 108 can comprise between one and twenty control wells with all other wells being test wells. In other example embodiments, the well plate 108 can comprise more than twenty control wells.

The wells 106 of the well plate 108 comprising the antibiotic (the test wells) can have the antibiotic already present within the wells 106. For example, such wells 106 can be pre-loaded with the antibiotic.

In some embodiments, the antibiotic within the test wells can be lyophilized or dried. For example, the antibiotic within the test wells can be in the form of a lyophilized disk, pellet(s), or powder.

In certain embodiments, the antibiotic within the test wells can be in aqueous form.

In some embodiments, the antibiotic can be added or introduced into the test wells prior to introducing the aliquots of the diluted sample 102.

In some embodiments, each well plate 108 can comprise multiple antibiotics such that some of the wells 106 of the well plate 108 are dedicated to a specific antibiotic and the other wells 106 of the well plate 108 are dedicated to one or more other antibiotics. In other embodiments, one well plate 108 can comprise only one type of antibiotic such that all test wells of the well plate 108 are dedicated to that one antibiotic.

In some embodiments, the test wells can comprise a bacteriostatic antibiotic, a bactericidal antibiotic, or a combination thereof.

In certain embodiments, the test wells can comprise a beta-lactam antibiotic (including, but not limited to, penicillins such as ampicillin, amoxicillin, flucloxacillin, penicillin, amoxicillin/clavulanate, and ticarcillin/clavulanate and monobactams such as aztreonam) or β-lactam and β-lactam inhibitor combinations (including, but not limited to, piperacillin-tazobactam and ampicillin-sulbactam).

In some embodiments, the test wells can comprise any of the following types of antibiotics: Aminoglycosides (including but not limited to amikacin, gentamicin, kanamycin, neomycin, netilmicin, paromomycin, streptomycin, spectinomycin, and tobramycin), Ansamycins (including but not limited to rifaximin), Beta-lactam combination agents (including but not limited to piperacillin-tazobactam, ampicillin-sulbactam, meropenem-varborbactam, imipenem-relebactam, sulbactam-durlobactam, ceftazidime-avibactam, ceftolozane-tazobactam), Carbapenems (including but not limited to ertapenem, doripenem, imipenem, and meropenem), Cephalosporins (including but not limited to ceftaroline, cefepime, ceftazidime, ceftriaxone, cefadroxil, cefalotin, cefazolin, cephalexin, cefaclor, cefprozil, fecluroxime, cefixime, cefdinir, cefditoren, cefotaxime, cefpodoxime, ceftibuten, and ceftobiprole), Chloramphenicols, Glycopeptides (including but not limited to vancomycin, teicoplanin, telavancin, dalbavancin, and oritavancin), Folate Synthesis Inhibitors (including but not limited to trimethoprim-sulfamethoxazole), Fluoroquinolones (including but not limited to ciprofloxacin), Lincosamides (including but not limited to clindamycin, lincomycin, azithromycin, clarithromycin, dirithromycin, roxithromycin, telithromycin, and spiramycin), Lincosamines, Lipopeptides, Macrolides (including but not limited to erythromycin), Monobactams, Nitrofurans (including but not limited to furazolidone and nitrofurantoin), Oxazolidinones (including but not limited to linezolid, posizolid, radezolid, and torezolid), Quinolones (including but not limited to enoxacin, gatifloxacin, gemifloxacin, levofloxacin, lomefloxacin, moxifloxacin, naldixic acid, norfloxacin, trovafloxacin, grepafloxacin, sparfloxacin, and temafloxacin), Rifampins, Streptogramins, Sulfonamides (including but not limited to mafenide, sulfacetamide, sulfadiazine, sulfadimethoxine, sulfamethizole, sulfamethoxazole, sulfasalazine, and sulfisoxazole), Tetracyclines (including but not limited to oxycycline, minocycline, demeclocycline, doxycycline, oxytetracycline, and tetracycline), polypeptides (including but not limited to bacitracin, polymyxin B, colistin, and cyclic lipopeptides such as daptomycin), phages, or a combination or derivative thereof.

In other embodiments, the test wells can comprise any of the following types of antibiotics: clofazimine, ethambutol, isoniazid, rifampicin, arsphenamine, chloramphenicol, cefiderocol, fosfomycin, metronidazole, tigecycline, trimethoprim, or a combination or derivative thereof.

For example, Table 3 below lists certain antibiotics for certain gram-negative and gram-positive bacteria that can be used for antibiotic susceptibility testing using the method 100, system, and devices disclosed herein:

TABLE 3
Gram-negative and gram-positive antibiotics
used for antimicrobial susceptibility testing

| Gram-negative antibiotics | Gram-positive antibiotics |
|---|---|
| Amikacin | Ampicillin |
| Ampicillin | Cefoxitin |
| Ampicillin-sulbactam | Ceftaroline |
| Aztreonam | Clindamycin |
| Cefazolin | Daptomycin |
| Cefepime | Doxycycline |
| Ceftazidime | Erythromycin |
| Ceftriaxone | High Level Gentamicin |
| Ceftolozane-tazobactam | High Level Streptomycin |
| Ceftazidime-avibactam | Linezolid |
| Ciprofloxacin | Oxacillin |
| Ertapenem | Penicillin |
| Gentamicin | Tedizolid |
| Imipenem | Tetracycline |
| Imipenem-relebactam | Trimethoprim-sulfamethoxazole |
| Levofloxacin | Vancomycin |
| Meropenem | |
| Meropenem-vaborbactam | |
| Piperacillin-tazobactam | |
| Tobramycin | |
| Trimethoprim-sulfamethoxazole | |
| Sulbactam-durlobactam | |

Also, for example, Table 4 below lists certain classes of antibiotics and specific examples of antibiotics that can be used for antibiotic susceptibility testing using the method 100, system, and devices disclosed herein:

TABLE 4
Classes of antibiotics and specific antibiotics
used for antimicrobial susceptibility testing

| Antibiotic | Class of Antibiotic |
|---|---|
| Amikacin | Aminoglycoside |
| Tobramycin | Aminoglycoside |
| Ampicillin-sulbactam | Beta-lactam combination agent |
| Ceftazidime-avibactam | Beta-lactam combination agent |
| Ceftolozane-tazobactam | Beta-lactam combination agent |
| Piperacillin-tazobactam | Beta-lactam combination agent |
| Imipenem | Carbapenem |
| Meropenem | Carbapenem |
| Cefazolin | Cephalosporin I |
| Ceftazidime | Cephalosporin III |
| Ceftriaxone | Cephalosporin III |
| Cefepime | Cephalosporin IV |
| Ciprofloxacin | Fluoroquinolone |
| Trimethoprim- sulfamethoxazole | Folate pathway antagonist |
| Aztreonam | Monobactam |

The method 100 can further comprise placing the sensor array lid 112 on top of the well plate 108 filled with the aliquots of the sample 102 or covering the well plate 108 filled with the aliquots of the sample 102 with the sensor array lid 112 in step 100C.

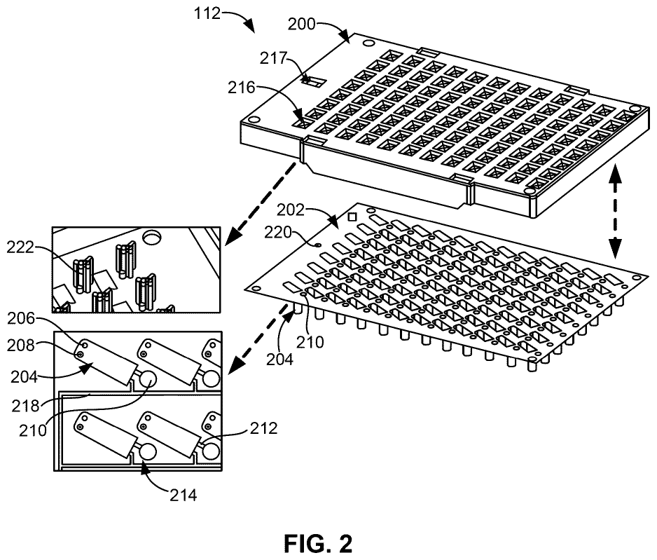

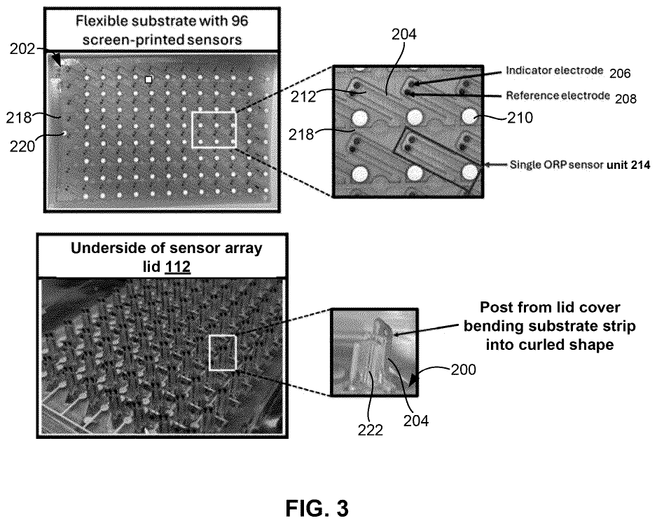

As will be discussed in more detail in relation to FIGS. 2 and 3, the sensor array lid 112 can comprise a plurality of sensor units 214 (see, e.g., FIGS. 2 and 3) extending from an underside of the sensor array lid 112. Each of the sensor units 214 can be configured to extend into a well 106 of the well plate 108 such that the sensor units 214 are at least partially immersed in the aliquots of the sample 102 within the wells 106 when the sensor array lid 112 is placed on top of the well plate 108 or covers the well plate 108. When the sensor array lid 112 is placed on top of the well plate 108 or covers the well plate 108, the assembled components can be collectively referred to as the testing device 110.

The sensor array lid 112 can be made of a clear polymeric material to allow a medical or laboratory professional or technician to view the wells 106 of the well plate 108 and to view the aliquots of the sample 102 within the wells 106 during the AST procedure. Moreover, the sensor array lid 112 can be made of a clear polymeric material to allow a medical or laboratory professional or technician to view the sensor units 214 and to ensure that the sensor units 214 are immersed in the aliquot of the sample 102 within the wells 106.

In some embodiments, the sensor array lid 112 can be made in part of at least one of polystyrene, polypropylene, a cyclic olefin copolymer, or another biocompatible polymeric material.

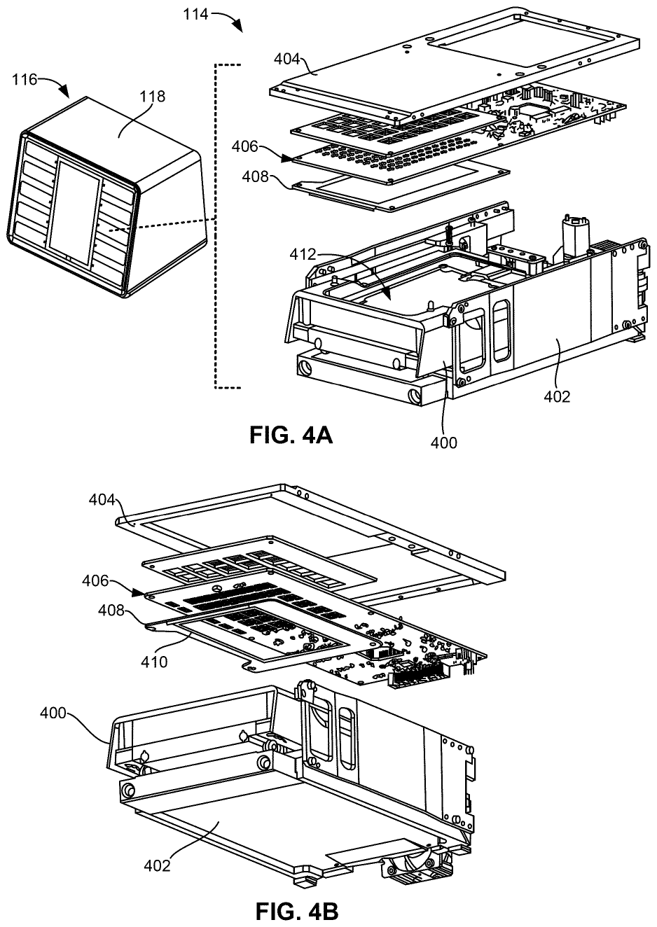

The method 100 can also comprise placing the testing device 110 (i.e., the well plate 108 covered by the sensor array lid 112) on a plate tray 400 (see, e.g., FIG. 4A) of a reader module 114 of a reader 116 and inserting the plate tray 400 carrying the testing device 110 into the reader 116 in step 100D.

The testing device 110 and the reader 116 can be part of a system to determine the susceptibility of bacteria to an antibiotic.

The reader 116 can comprise a reader housing 118 or exterior housing configured to house a plurality of reader modules 114. At least part of each of the reader modules 114 can be automatically driven, pushed, or otherwise translated at least partially out of the reader housing 118 to receive the testing device 110. For example, each of the reader modules 114 can comprise a tray carrier 402 (see, e.g., FIGS. 4A and 4B) configured to automatically push, drive, or otherwise translate the plate tray 400 at least partially out of the reader housing 118 to receive the testing device 110 (i.e., the well plate 108 covered by the sensor array lid 112).

Once the testing device 110 is placed on the plate tray 400, the method 100 can also comprise pushing the plate tray 400 or part of the tray carrier 402 back into the reader housing 118 or causing the plate tray 400 or part of the tray carrier 402 to be retracted into the reader housing 118.

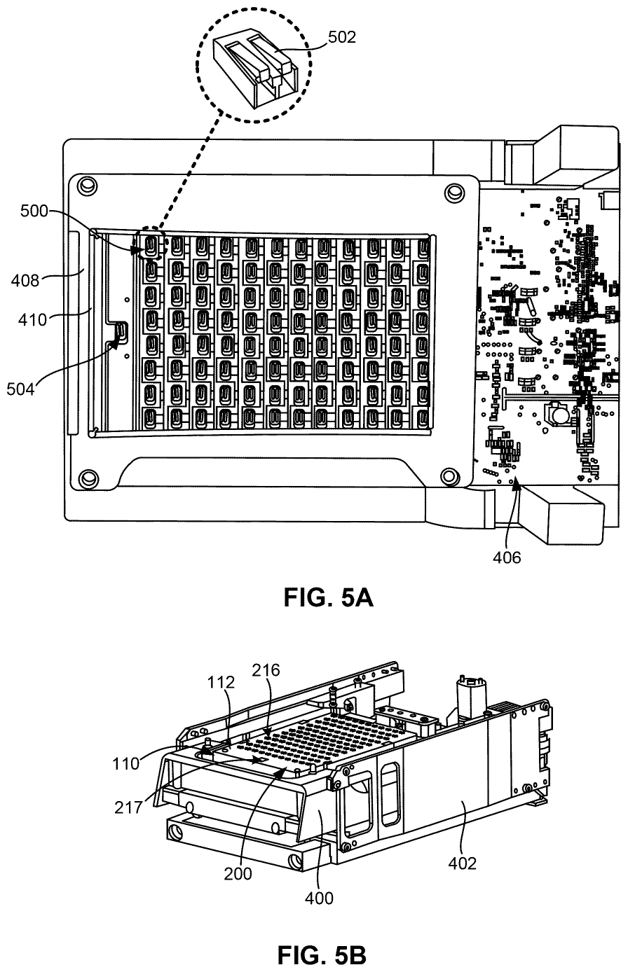

As will be discussed in more detail in relation to FIGS. 4A, 4B, and 5A, each of the reader modules 114 can also comprise a printed circuit board assembly (PCBA) 406 disposed above the plate tray 400 when the plate tray 400 is retracted back into the reader housing 118. The PCBA 406 can comprise a plurality of conductive connectors 500.

The conductive connectors 500 can extend downward from an underside of the PCBA 406 to contact conductive pads 210 of the sensor array lid 112 when the sensor array lid 112 covers the well plate 108 placed on the plate tray 400. As previously discussed, the sensor units 214 of the sensor array lid 112 can be configured to be immersed in the aliquots of the sample 102 within the wells 106 of the well plate 108.

The reader 116 can further comprise one or more processors or processing units and one or more memory units. In some embodiments, the one or more processors and the one or more memory units can refer to processors and memory units of the PCBA 406. In certain embodiments, the one or more processors and the one or more memory units can refer to processors and memory units of one or more microcontroller units (MCUs) within the reader 116. In other embodiments, the one or more processors and the one or more memory units can refer to processors and memory units of a single board computer (SBC) housed within the reader housing 118.

In some embodiments, each of the reader modules 114 can comprise its own MCU. Each of the MCUs can be programmed to transmit raw digitized signals to the SBC via a serial interface (e.g., RS-485).

The one or more processors of the reader 116 (e.g., the one or more processors of the SBC) can be programmed to determine the susceptibility of the bacteria to the antibiotic based on any changes in a solution characteristic of the aliquots of the sample 102 within the test wells of the well plate 108 compared to the control well over time.

In some embodiments, the solution characteristic monitored by the reader 116 can be the oxidation reduction potential (ORP) of the aliquots of the sample 102 within the wells 106 of the well plate 108.

Oxidation reduction potential (ORP) can refer to the proportion of oxidized molecules to reduced molecules in the aliquots of the sample 102 and is an effective metric for monitoring for bacterial growth and metabolism (or lack thereof). Oxygen and other electron donors are consumed when the bacteria within the aliquots of the sample 102 grow and metabolize. This results in a higher proportion of reduced molecules and hence a more negative ORP.

The reader 116 can act as a high-impedance voltmeter to measure a potential difference between an indicator or active electrode 206 (see, e.g., FIG. 2 or FIG. 3) of one of the sensor units 214 immersed in an aliquot of the sample 102 within one of the wells 106 and a reference electrode 208 or pseudo reference electrode (see, e.g., FIG. 2 or FIG. 3) of the same sensor unit 214 immersed in the aliquot of the sample 102.

The active electrode 206 of each of the sensor units 214 can be electrically connected or otherwise electrically coupled to a conductive pad 210 of the sensor unit 214 via conductive traces 212 (see, e.g., FIG. 2 or FIG. 3). As will be discussed in more detail in relation to FIGS. 2 and 3, the conductive traces 212 can be routed along one side of a sensor substrate layer 202 or along both sides of the sensor substrate layer 202. In certain embodiments, the conductive traces 212 can be routed through the body of the sensor substrate layer 202.

As previously discussed, the conductive connectors 500 within each of the reader modules 114 can be configured to electrically contact or engage with the conductive pads 210 of the sensor array lid 112 of the testing device 110 once the testing device 110 is within the reader housing 118 (i.e., once the plate tray 400 carrying the testing device 110 is retracted or pushed back into the reader housing 118).

The reader 116 can automatically begin to read signals from the sensor units 214 of the sensor array lid 112 once the testing device 110 is inserted into the reader 116. The reader 116 can be configured to read signals from the sensor units 214 in order to detect any changes in the solution characteristic of the aliquots of the sample 102 within the wells 106 of the well plate 108. Since the test wells of the well plate 108 contain antibiotics, the reader 116 can be used to determine the susceptibility of the bacteria within the sample 102 to the antibiotics.

As will be discussed in more detail in the following sections, the reader 116 can also comprise one or more heating components, temperature sensors, humidity sensors, and sealing components to optimize bacterial growth conditions within each of the reader modules 114.

For example, each of the reader modules 114 can further comprise a gasket 410 (see, e.g., FIGS. 4B, 5A, or 6). The gasket 410 can be configured to create a partial seal around the top of the sensor array lid 112 and the bottom of the PCBA 406 to control an evaporation rate of the aliquots of the sample 102 and to control a humidity level and a partial pressure of oxygen within a headspace above the well plate 108.

The method 100 can also comprise heating the conductive connectors 500 of the PCBA 406 above a dew point using an upper heater to control condensation on the conductive connectors. In some embodiments, the upper heater can be coupled to or integrated within the PCBA 406. In some embodiments, the upper heater can refer to a heater resistance trace that is located as an interlayer inside of the PCBA 406.

In some embodiments, the method 100 can also comprise monitoring a temperature and a humidity level within a headspace above the well plate 108 using a temperature sensor and a humidity sensor. For example, the headspace can comprise a volume of about 9,012 cubic millimeters. The size of the headspace can be adjusted based on the size of the gasket 410, the reader module 114, the well plate 108, the sensor array lid 112, or a combination thereof.

The method 100 can also comprise heating the well plate 108 using a lower heater 412 (see, e.g., FIG. 4A) coupled to the plate tray 400 to ensure optimal bacterial growth conditions. The lower heater 412 can heat the well plate 108 to between about 30° C. and about 40° C.

In some embodiments, the method 100 can further comprise conducting certain internal quality checks prior to or while determining the susceptibility of the bacteria to the antibiotic. For example, the method 100 can comprise checking, using the one or more processors, that a baseline voltage of a starting ORP of each of the aliquots of the sample 102 (contained within the wells 106 of the well plate 108) is between about 2600 millivolts (mV) and 2750 mV (e.g., between about 100 mV and 250 mV, plus the 2.5V DC offset). Also, for example, the internal quality check can comprise checking, using the one or more processors, that a voltage noise of each of the sensor units 214 is between about 0 millivolt root-mean-square (mVrms) and 5 mVrms. In addition, the internal quality check can further comprise checking, using the one or more processors, that a sensor voltage drift of each of the sensors is between about 0 mV per hour and 40 mV per hour. In these embodiments, the sensor units 214 are ORP sensors.

The reader 116 can analyze the signals obtained from the plurality of sensor units 214 of the testing device 110 and provide information concerning the susceptibility of the bacteria to the antibiotic (e.g., levels of susceptibility), along with information concerning minimum inhibitory concentrations (MICs).

As shown in FIG. 1, the reader 116 can also comprise a display 120. In some embodiments, the display 120 can be an interactive touchscreen display. The display 120 can render graphics, messages, or other types of text, or a combination thereof concerning the results of the antimicrobial susceptibility test. In certain embodiments, the display 120 can allow a user to input commands to the reader 116 concerning an upcoming test, an ongoing test, or a completed test. In certain embodiments, the reader 116 can display the MICs and the level(s) of susceptibility via the display 120 or convey the results of the testing procedure via one or more audible alerts or commands.

FIG. 2 illustrates certain components of a sensor array lid 112. The sensor array lid 112 can comprise a lid cover 200 and a sensor substrate layer 202 coupled to an underside of the lid cover 200. The lid cover 200 can have a concavity or accommodation space on the underside of the lid such that the sensor substrate layer 202 can reside within the concavity or accommodation space of the lid cover 200.

In some embodiments, the sensor substrate layer 202 can be made, at least in part, of a flexible polymeric material. For example, the sensor substrate layer 202 can be made in part of a flexible sheet of polyethylene terephthalate (PET). Also, for example, the sensor substrate layer 202 can also be made in part of a flexible printed circuit board (PCB) material. For example, the sensor substrate layer 202 can be made in part of polyimide or polyamide.

In alternative embodiments, the sensor substrate layer 202 can be made in part of a conductive metal substrate. For example, in these embodiments, the sensor substrate layer 202 can be made in part of a sheet of stainless steel foil.

The sensor substrate layer 202 can be coupled to the underside of the lid cover 200 by a biocompatible adhesive (e.g., a biocompatible polymeric adhesive, a cyanoacrylate adhesive, etc.) and/or a plurality of fasteners (e.g., screws, clips, clasps, etc.).

The sensor substrate layer 202 can comprise a plurality of substrate strips 204 or substrate segments partially cut out or otherwise separated from a remainder of the sensor substrate layer 202 (i.e., the parts of the sensor substrate layer 202 coupled to the underside of the sensor array lid 112). The substrate strips 204 can be curled or bent vertically downward relative to a surrounding portion of the sensor substrate layer 202. The substrate strips 204 can maintain its curled or bent configuration even when the sensor array lid 112 covers or caps the well plate 108.

In some embodiments, the entire sensor array lid 112 (including the lid cover 200 and the sensor substrate layer 202) can be made to be disposable or used only one time. In these embodiments, the sensor array lid 112 can be discarded after a testing procedure has been completed.

Each of the substrate strips 204 can comprise an active electrode 206 (also referred to as an indicator electrode) and a reference electrode 208 (or pseudo-reference electrode) disposed on the substrate strip 204.

The active electrode 206 can be electrically connected or coupled to a conductive pad 210 disposed on a top side of the sensor substrate layer 202. As shown in FIG. 2, the conductive pads 210 can be positioned or otherwise configured such that at least part of each of the conductive pads 210 is exposed by openings 216 defined along the lid cover 200.

The top side or top surface of the sensor substrate layer 202 can be a side or surface that faces or contacts an underside of the lid cover 200.

In some embodiments, each of the conductive pads 210 can be a conductive metallic disk that extends through the sensor substrate layer 202. In certain embodiments, the conductive pads 210 can be adhered to or screen-printed onto the sensor substrate layer 202.

The active electrodes 206 can each be electrically connected or coupled to one conductive pad 210 by a conductive trace 212.

In some embodiments, the conductive traces 212 can be silver or platinum traces or routing lines. In other embodiments, the conductive traces 212 can be made of another conductive material such as gold, copper, etc.

For purposes of this disclosure, each of the substrate strips 204, comprising the active electrode 206, the reference electrode 208, and the conductive pad 210 can collectively be referred to as a sensor unit 214.

In some embodiments, all of the reference electrodes 208 (or pseudo-reference electrodes) can be electrically connected or otherwise coupled to a singular electrical contact pad 220 disposed at one end, corner, or edge of the sensor substrate layer 202. All of the reference electrodes 208 can be electrically connected or otherwise coupled to the singular electrical contact pad 220 via additional conductive traces 218 routed along a surface or side of the sensor substrate layer 202, through the body of the sensor substrate layer 202, and/or through a body of the lid cover 200.

One function of the lid cover 200 of the sensor array lid 112 can be to protect the aliquots of the sample within the wells 106 from contamination. Another function of the lid cover 200 can be to prevent the aliquots of the sample from spilling out or inadvertently leaking. Yet another function of the lid cover 200 can be to serve as an interface to facilitate alignment of the conductive connectors 500 of the PCBA 406 with the conductive pads 210 of the sensor substrate layer 202.

As shown in FIG. 2, the lid cover 200 can comprise a plurality of openings 216 that are configured to expose the conductive pads 210. The plurality of openings 216 can each correspond to one of the plurality of conductive connectors 500. The plurality of openings 216 can allow the conductive connectors 500 to extend past the lid cover 200 to contact the conductive pads 210 of the sensor substrate layer 202.

The lid cover 200 can also comprise an additional opening 217 configured to expose the singular electrical contact pad 220 electrically connected to the reference electrodes 208. The additional opening 217 can be positioned to align with the location of the singular electrical contact pad 220.

Each of the conductive connectors 500 of the PCBA 406 can comprise one or more conductive contacts (e.g., two spring leaf contacts) to contact each of the conductive pads 210 in order to measure a change in a solution characteristic (e.g., ORP) of the aliquot of the sample 102 within each of the wells 106.

As will be discussed in more detail in later sections, the PCBA 406 can also comprise a reference conductive connector 504 (see FIG. 5A). The reference conductive connector 504 can extend through the additional opening 217 to electrically contact the singular electrical contact pad 220 connected to the reference electrodes 208.

The testing device 110 (see, e.g., FIG. 1), in the assembled configuration, can have a device length, a device width, and a device height. In some embodiments, the testing device 110 in the assembled configuration can have a device length of between about 80.0 mm and 160.0 mm (e.g., about 122.5 mm), a device width of between about 60.0 mm and 100.0 mm (e.g., 81.0 mm), and a device height of between about 10.0 mm and 30.0 mm (e.g., about 20.0 mm).

Each of the sensor units 214 (implemented, for example, as curled or bent substrate strips 204) can extend into a well 106 of the well plate 108 when the sensor array lid 112 covers or caps the well plate 108. When the wells 106 of the well plate 108 are filled with an aliquot of the sample 102 (also referred to as the inoculum), at least part of the sensor unit 214 can be immersed in the aliquot of the sample 102.

In some embodiments, the sensor units 214 are implemented as curled or bent substrate strips 204 extending downward from the sensor substrate layer 202. Each of the substrate strips 204 can comprise an active electrode 206 and a reference electrode 208 printed, deposited, or electroplated onto a distal end or distal portion of the substrate strip 204.

The substrate strips 204 can extend into the wells 106 of the well plate 108. When the wells 106 of the well plate 108 are filled with aliquots of the sample 102 (e.g., a positive blood culture), at least a distal segment or portion of each of the substrate strips 204 (the distal segment or distal portion comprising the active electrode 206 and the reference electrode 208) can be immersed in the aliquot of the sample 102.

The sensor units 214 can be aligned to match the alignment or arrangement of the wells 106. For example, when the well plate 108 comprises 96 wells arranged as an 8×12 array of wells 106, the sensor array lid 112 can comprise 96 sensor units 214 arranged as an 8×12 array of sensor units 214.

In some embodiments the sensor units 214 can be spaced between about 6.00 mm and 12.0 mm (about 9.00 mm) apart from each other.

In certain embodiments, the sensor units 214 can be arranged in such a way that none of the sensor units 214 touch or make contact with the walls of the wells 106 when the sensor array lid 112 covers or caps the well plate 108.

In other embodiments, the sensor units 214 can be arranged in such a way that the sensor units 214 rest against or makes contact with one or more walls of the wells 106 when the sensor array lid 112 covers or caps the well plate 108.

When the sensor units 214 are implemented as substrate strips 204, the sensor array lid 112 can comprise a plurality of posts 222 (e.g., polymeric posts or protrusions) extending from the underside of the lid cover 200. The posts 222 can be configured to push or press against the substrate strips 204 such that the substrate strips 204 maintain their curled or bent configuration.

For example, the posts 222 can be angled to allow the posts 222 to push or press against the substrate strips 204 to ensure the substrate strips 204 maintain their curled or bent configuration. As a more specific example, the posts 222 can be positioned at an oblique angle with respect to the underside of the lid top.

In some embodiments, the posts 222 can be comprised of one or more individual posts that come together to form a combined single post. In some embodiments, the individual posts can be substantially shaped as flattened rectangular posts and the combined single post can be shaped as an elongated Y-shape or X-shape.

The sensor substrate layer 202 can further comprise a singular electrical contact pad 220 disposed at one end, corner, or edge of the sensor substrate layer 202. The singular electrical contact pad 220 can be coupled to a plurality of additional conductive traces 218 that are each connected to a reference electrode 208 of a sensor unit 214. In some embodiments, the additional conductive traces 218 can be routed along a surface or side of the sensor substrate layer 202 (e.g., the side or surface adhered or coupled to the underside of the lid top). In other embodiments, the additional conductive traces 218 can be routed or can extend through the sensor substrate layer 202 or through a body of the lid cover 200. For example, when the sensor substrate layer 202 is made of a PCB material, the additional conductive traces 218 can be routed or directed through vias or through-holes arranged along the sensor substrate layer 202.

The singular electrical contact pad 220 can be configured to contact or otherwise engage with a reference conductive connector 504 of the PCBA 406 (see, e.g., FIG. 5A) when the testing device 110 (in the assembled configuration) is inserted or introduced into a receiving slot of the reader 116 to allow the reader 116 to obtain signals from the sensor units 214 of the testing device 110.

FIG. 3 illustrates a sensor substrate layer 202 comprising a plurality of screen-printed sensor units 214 making up part of the sensor array lid 112 and an underside of the sensor array lid 112 with posts 222 extending from an underside of the lid cover 200 to bend the substrate strips 204 into position into their respective well 106.

Each post 222 can push against a substrate strip 204 to allow the substrate strip 204 to maintain or configure into a curled or bent configuration. The post 222 can push or press against the substrate strip 204 in such a way that a distal segment of the substrate strip 204 (for example, the distal segment comprising the active electrode 206 and the reference electrode 208) is substantially perpendicular to portions of the sensor substrate layer 202 that are coupled to the underside of the lid cover 200.

The substrate strips 204 can be positioned in rows and columns and at an angle with respect to an edge of sensor substrate layer 202. In some variations, the substrate strips 204 can be positioned perpendicular or parallel to an edge of the sensor substrate layer 202.

In some embodiments, the post 222 can push or press against the substrate strip 204 in such a way that the distal segment of the substrate strip 204 is positioned at an oblique angle (more specifically, an angle between 60° and 90°) with respect to portions of the sensor substrate layer 202 that are coupled to the underside of the lid cover 200.

In some embodiments, the substrate strip 204 can be formed by cutting the sensor substrate layer 202 along the three sides surrounding the substrate strip 204.

In some embodiments, the substrate strips 204 can be substantially rectangular in shape. For example, the substrate strips 204 can be formed as rectangular tabs or rectangular strips.

In other embodiments, the substrate strips 204 can be substantially triangular, oval, or semicircular in shape. In further embodiments, the substrate strips 204 can be shaped as leaves or leaflets.

As discussed with reference to FIG. 2, each of the substrate strips 204 can comprise an active electrode 206, a reference electrode 208, and a conductive pad 210. The active electrode 206 and the reference electrode 208 can be placed towards the end of the substrate strip 204.

In some embodiments, the conductive pads 210 can be made of silver. In other embodiments, the conductive pads 210 can be made of another conductive material such as gold, copper, aluminum, nickel, etc.

In these and other embodiments, the posts 222 can be made of the same non-conductive material (e.g., polymeric material) used to make the lid cover 200.

In some embodiments, the posts 222 can be rods or pins extending from the underside of the lid cover 200. In further embodiments, the posts 222 can be adhered to or otherwise fastened to the underside of the lid cover 200. In certain embodiments, the posts 222 can be replaced by protuberances or other type of surface features protruding from the underside of the lid cover 200.

Although the figures illustrate the substrate strips 204 being pushed or pressed into the curled or bent configuration by the posts 222, it is contemplated by this disclosure that the substrate strips 204 can also attain and maintain their curled or bent configuration without the assistance of the posts 222 (e.g., by being pre-shaped, pre-set, pre-trained, or otherwise manipulated into such a configuration).

The distal segment of the sensor unit 214 can comprise the active electrode 206 and the reference electrode 208 disposed on the substrate strip 204. The wells 106 of the well plate 108 can be sized to hold a sufficient amount of the aliquot of the sample 102 to allow at least the distal segment of the sensor unit 214 to be immersed in the aliquot of the sample 102 when the sensor array lid 112 covers or caps the well plate 108.

At least one of the active electrode 206 and the reference electrode 208 can be screen-printed, electroplated, or sputter deposited on the substrate strip 204.

The active electrode 206 can comprise or be made of a redox-active material. In some embodiments, the redox-active material can be a noble metal. For example the redox-active material can be platinum, gold, or a combination or alloy thereof. In other embodiments, the redox-active material can be a redox-sensitive metal oxide.

In other embodiments, the redox-active material can be a conductive metal oxide such as iridium oxide, ruthenium oxide, or any combination or alloys of such materials with noble metals. In additional embodiments, the redox-active material can be a carbon-based electrode.

The reference electrode 208 can comprise or be made of a reference electrode material. In some embodiments, the reference electrode material can comprise at least one of silver/silver chloride (Ag/AgCl) and carbon. For example, when the reference electrode material is Ag/AgCl or carbon, the reference electrode material can be screen-printed onto the substrate strip 204.

The reference electrode 208 can be considered a pseudo-reference electrode since the reference electrode 208 operates without a reference buffer. A pseudo-reference electrode can be used in these instances since measurements are made by comparing changes in the signal rather than comparing absolute values. The reference electrode material can be coated by an ion exchange membrane or an ionomer coating.

In some embodiments, the reference electrode material of the reference electrode 208 can be screen-printed onto the sensor substrate layer 202. In these and other embodiments, the redox-active material of the active electrode 206 can also be screen-printed onto the sensor substrate layer 202.

In additional embodiments, an ion exchange membrane or an ionomer coating can also be screen-printed onto at least part of the sensor substrate layer 202. For example, an ion exchange membrane or an ionomer coating can be screen-printed onto a reference electrode material (e.g., Ag/AgCl or carbon/graphite) that has already been screen-printed onto the sensor substrate layer 202.

The redox-active material can be a noble metal such as platinum, gold, or a combination or alloy thereof. In these embodiments, the redox-active material can initially take the form of an ink or paste (e.g., platinum or gold ink or paste). The ink or paste (e.g., platinum or gold ink or paste) can be screen-printed onto the sensor substrate layer 202 using the method previously disclosed.

In other embodiments, the redox-active material can be a conductive metal oxide such as iridium oxide, ruthenium oxide, or any combination or alloys of such materials with noble metals. In additional embodiments, the redox-active material can be a carbon-based electrode.

The reference electrode material can also initially take the form of an ink or paste (e.g., silver/silver chloride or graphite ink or paste). This ink or paste (e.g., silver/silver chloride or graphite ink or paste) can be screen-printed onto another portion of the sensor substrate layer 202.

For example, the redox-active material can be screen-printed onto a distal segment of a substrate strip 204. In this example, the reference electrode material can be screen-printed onto this same distal segment of the substrate strip 204 but next to or in proximity to the redox-active material.

In alternative embodiments, the Ag/AgCl reference electrode material can also be made by chlorinating silver with electrical current flow in a chlorinated solution.

In some embodiments, a reference electrode material can be printed onto the sensor substrate layer 202 and the reference electrode material can be covered entirely by an ion exchange membrane.

In some embodiments, the ion exchange membrane can be an ionomer coating capable of blocking certain ions (e.g., Ag⁺ ions) that can interact with or adversely affect certain microbial organisms or other infectious agents. For example, the ion exchange membrane can be a sulfonated tetrafluoroethylene based fluoropolymer-copolymer such as Nafion™. The sulfonated tetrafluoroethylene based fluoropolymer-copolymer can also be referred to as a proton exchange membrane since it can be designed to only allow positively charged ions (e.g., H⁺ ions) to freely flow through its polymer layer but can slow the diffusion or flow of other ions (e.g., Ag⁺ ions) that may interact with or be harmful to certain microbial organisms or other infectious agents, keeping such ions close to the reference electrode material.

In other embodiments, the ion exchange membrane can be a polyaromatic polymer anion exchange membrane such as Fumion™. The polyaromatic polymer anion exchange membrane can be designed to only allow anions to pass through its polymer layer. Since certain harmful ions such as Ag⁺ ions are cations, such ions are blocked from entering the sample.

In some embodiments, the ion exchange membrane can be screen-printed onto the sensor substrate layer 202 and onto the reference electrode material disposed on the sensor substrate layer 202. In certain embodiments, the reference electrode material can first be screen-printed onto the sensor substrate layer 202 and the ion exchange membrane can be subsequently screen-printed onto the reference electrode material and part of the sensor substrate layer 202.

In alternative embodiments, at least one of the active electrode 206 and the reference electrode 208 can be formed via sputter deposition.

In alternative embodiments, at least one of the active electrode 206 and the reference electrode 208 can be formed via electroplating.

FIGS. 4A and 4B are exploded views illustrating components of a reader module 114 of the reader 116. Each of the reader modules 114 can comprise a moveable or slidable plate tray 400 held or carried by a tray carrier 402. The plate tray 400 can be configured and sized to receive and hold an assembled testing device 110 comprising the well plate 108 covered by the sensor array lid 112 (see FIG. 1). Each of the reader modules 114 can also comprise a printed circuit board assembly (PCBA) 406 disposed above the plate tray 400 when the plate tray 400 is inserted or otherwise retracted into a reader housing 118 of the reader 116. The reader housing 118 can house and contain all of the reader modules 114 and separate each of the reader modules 114 from one another. In some embodiments, the reader housing 118 of the reader 116 can comprise between four reader modules 114 and ten reader modules 114 (e.g., any of four reader modules 114, five reader modules 114, six reader modules 114, seven reader modules 114, eight reader modules 114, nine reader modules 114, or ten reader modules 114).

The reader module 114 can comprise a module cover 404 configured to couple with the tray carrier 402 to form the top and bottom of the reader module 114. The module cover 404 can partially cover the top of the PCBA 406. The module cover 404 can also comprise an opening for certain electrical components of the PCBA 406 to interface with electrical components within the reader 116.

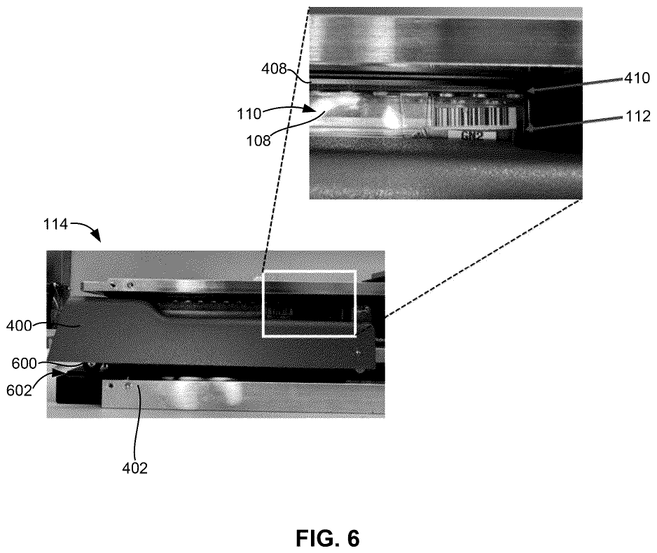

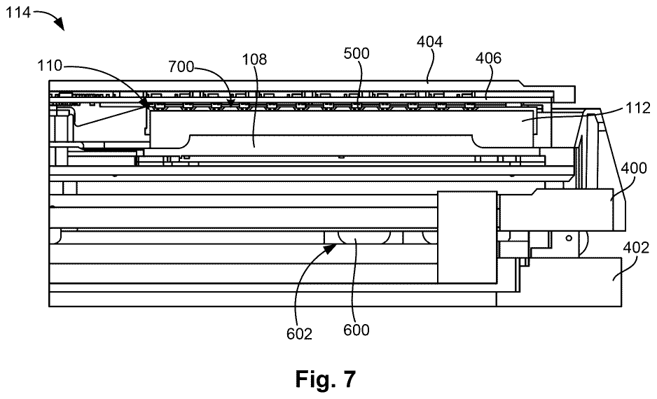

Each of the tray carriers 402 can be coupled to the reader housing 118. Each of the tray carriers 402 can be configured to hold a moveable, slidable, or translatable plate tray 400. In some embodiments, the plate tray 400 can comprise wheels 600 coupled to a bottom or the exterior lateral sides of the plate tray 400 and the tray carrier 402 can comprise wheel tracks 602 for interfacing with the wheels 600 of the plate tray 400 (see FIGS. 6 and 7). The wheels 600 of the plate tray 400 can slide or roll along the wheel tracks 602 of the tray carrier 402. Each of the reader modules 114 can also comprise one or more motors (e.g., servo motors) configured to control the translation or movement of the plate tray 400 into or out of the reader housing 118.

In some embodiments, each of the reader modules 114 can further comprise a gasket plate 408 disposed in between the bottom of the PCBA 406 and the top of the sensor array lid 112. The gasket plate 408 can comprise a gasket 410 adhered or otherwise coupled to an underside of the gasket plate 408.

When the gasket 410 is positioned over the top of the sensor array lid 112, the gasket 410 can create a partial seal around the top of the lid cover 200 of the sensor array lid 112. The gasket 410 can also act as a perimeter surrounding a plurality of conductive connectors 500 extending downward from the underside of the PCBA 406.

The gasket 410 can be made in part of a semipermeable polymeric material having a Shore A hardness of between about 20 to 55 (e.g., a Shore A hardness of about 50). In some embodiments, the gasket 410 can be made of polysiloxane. For example, the gasket 410 can be made of silicone rubber.

In some embodiments, the gasket 410 can be shaped as a rectangle. In other embodiments, the gasket 410 can be shaped as a square or another polygonal shape, an oval, or a circle.

In some embodiments, the gasket 410 can have a gasket thickness of between about 0.50 mm and 0.90 mm. For example, the gasket 410 can have a gasket thickness of about 0.80 mm.

One technical problem faced by the applicant is how to control undesirable sample evaporation but still allow for some air mass transfer to replenish the partial pressure of oxygen in the test headspace 700 (see, e.g., FIG. 7) above the sensor array lid 112. One technical solution discovered and/or developed by the applicant is the gasket 410 disclosed herein which is made of a semipermeable polymeric material such as silicone rubber and having a Shore A hardness of between about 20 to 55 (e.g., a Shore A hardness of about 50). The gasket 410, exhibiting the durometer disclosed herein and when compressed in between the bottom of the gasket plate 408 and the top of the lid cover 200 of the sensor array lid 112, creates a partial seal around the test headspace 700 above the assembled testing device 110 and allows for just the right amount of oxygen to be let into the test headspace 700 to replenish the oxygen used by the bacteria within the wells 106 but prevents undesirable evaporation of the aliquots of the sample within the wells 106. Since the gasket 410 also helps create part of the test headspace 700, the gasket 410 plays a part in creating a controlled environment within each of the reader modules 114 that can be optimized for bacterial growth.

The PCBA 406 can be positioned between the gasket plate 408 and the module cover 404. The PCBA 406 can comprise an upper heater integrated with the PCBA 406. The upper heater can be configured to heat the plurality of conductive connectors 500 and the reference conductive connector 504 of the PCBA 406 above the dew point to prevent condensation from forming on the conductive connectors. In some embodiments, the upper heater can refer to a heater resistance trace that is located as an interlayer inside of the PCBA 406.

One technical benefit to heating the conductive connectors of the PCBA 406 is to prevent condensation from forming on the connectors since condensation might interfere with the signal or cause damage to the connectors over time.

The reader module 114 can further comprise a temperature sensor and/or a humidity sensor positioned within the test headspace 700 to provide measurements that can be used to calculate the dew point and to ensure that the upper heater is heating the conductive connectors 500 to a temperature that controls for condensation.

In some embodiments, each of the reader modules 114 can further comprise a lower heater 412 coupled to the plate tray 400. The well plate 108 of the testing device 110 can be configured to be placed above the lower heater 412. The lower heater 412 can be configured to heat the well plate 108 to ensure optimal bacterial growth conditions within the wells 106 of the well plate 108.

The lower heater 412 can be a heat spreader that comprises one or more thermistors. The lower heater 412 can be made of a high thermal conductive metallic material for efficient heat dissipation. The heat spreader can be made of gold, nickel, copper, or a combination or alloy thereof. In some embodiments, the lower heater 412 can be a resistive heater. In other embodiments, the lower heater 412 can be a Peltier heater.

In some embodiments, the temperature of the upper heater can be set to be about 2° C. above the temperature of the lower heater 412. For example, the upper heater can be set to between about 32° C. and about 42° C. (e.g., about 37° C.) while the lower heater 412 can be set to between about 30° C. and about 40° C. (e.g., about 35° C.).

FIG. 5A illustrates a plurality of conductive connectors 500 extending from an underside of the PCBA 406. The PCBA 406 can be considered part of a top portion of the reader module 114.

FIG. 5B illustrates a bottom portion of the reader module 114 with an assembled testing device 110 placed on the plate tray 400 of the reader module 114. As previously discussed, part of a method of performing antibiotic susceptibility testing using the reader 116 and the testing device 110 can comprise pushing or otherwise inserting the plate tray 400 holding the assembled testing device 110 into the reader housing 118. When the plate tray 400 is fully inserted or otherwise retracted back into the reader housing 118 by the tray carrier 402, the conductive connectors 500 of the PCBA 406 can be placed in electrical contact with the conductive pads 210 of the sensor array lid 112. The conductive connectors 500 can extend downward from the underside of the PCBA 406 through the openings 216 defined along the lid cover 200 of the sensor array lid 112 to contact the conductive pads 210 (see, e.g., FIGS. 2 and 3).

In some embodiments, the conductive connectors 500 can be leaf spring connectors or spring compression connectors. The leaf spring connectors or spring compression connectors can be made in part of a conductive metal or metal alloy.

For example, the leaf spring connectors or the spring compression connectors can be made of a copper alloy. As a more specific example, the conductive connectors 500 can be spring connectors or compression connectors distributed by Bourns, Inc.

As shown in FIG. 5A, in some embodiments, each of the conductive connectors 500 can comprise two spring leaf contacts 502. Both of the spring leaf contacts 502 can make contact with one conductive pad 210 on the sensor array lid 112. The spring leaf contacts 502 can be made of a conductive metal or metal alloy (e.g., copper alloy) and can protrude at an angle (downward) relative to a circuit board of the PCBA 406. This can allow the spring leaf contacts 502 to more easily extend through the opening 216 to contact the conductive pad 210.

FIG. 5A also illustrates that the PCBA 406 can comprise a singular reference conductive connector 504. The reference conductive connector 504 can extend through the additional opening 217 of the lid cover 200 of the sensor array lid 112 to electrically contact the singular electrical contact pad 220 connected to the plurality of reference electrodes 208 of the testing device 110. The singular reference conductive connector 504 can be connected or otherwise electrically coupled to a reference voltage line of the reader 116.

The reference conductive connector 504 can also extend downward from the underside of the PCBA 406. The reference conductive connector 504 can be made of the same material as the conductive connectors 500. For example, the reference conductive connector 504 can also be a leaf spring connector or spring compression connector. As a more specific example, the leaf spring connector or spring compression connector can be made in part of a conductive metal or metal alloy (e.g., copper alloy).

The reference conductive connector 504 can also comprise spring leaf contacts (e.g., two spring leaf contacts) that protrude at an angle (downward) relative to the circuit board of the PCBA 406. This can allow the spring leaf contacts to more easily extend through the additional opening 217 to contact the singular electrical contact pad 220.

When all of the conductive connectors 500 are in electrical contact with the conductive pads 210 of the sensor array lid 112 and the reference conductive connector 504 is in electrical contact with the singular electrical contact pad 220, the circuit is completed and the reader 116 can act as a high-impedance voltmeter to measure a potential difference between the reference electrode 208 and each of the active electrodes 206.

One technical problem faced by the applicants is how to design a reader that can effectively read analog signals from a sensor array lid comprising numerous sensor units formed from flexible substrate strips partially immersed in aliquots of a sample within wells of a well plate. One technical solution discovered and/or developed by the applicants is the PCBA 406 disclosed herein comprising conductive connectors extending directly downward from the underside of the PCBA 406. The conductive connectors are leaf spring connectors or spring compression connectors that extend downward at an angle and contact the conductive pads of the sensor array lid through openings defined along a lid cover of the sensor array lid.

In some embodiments, each of the reader modules 114 can comprise one or more processors programmed to read out analog signals from the sensor units 214 of the testing device 110, digitize the signals, and then communicate the digitized signals to a microcontroller unit (MCU).

In some embodiments, the analog signals read from the sensor units 214 can first be buffered by a buffering circuit and then provided as inputs to an analog multiplexer (MUX) within the reader 116. The analog multiplexer can iterate over the plurality of sensor units 214 and testing devices 110. The analog signals can then be converted to digital signals for analysis by the MCU.

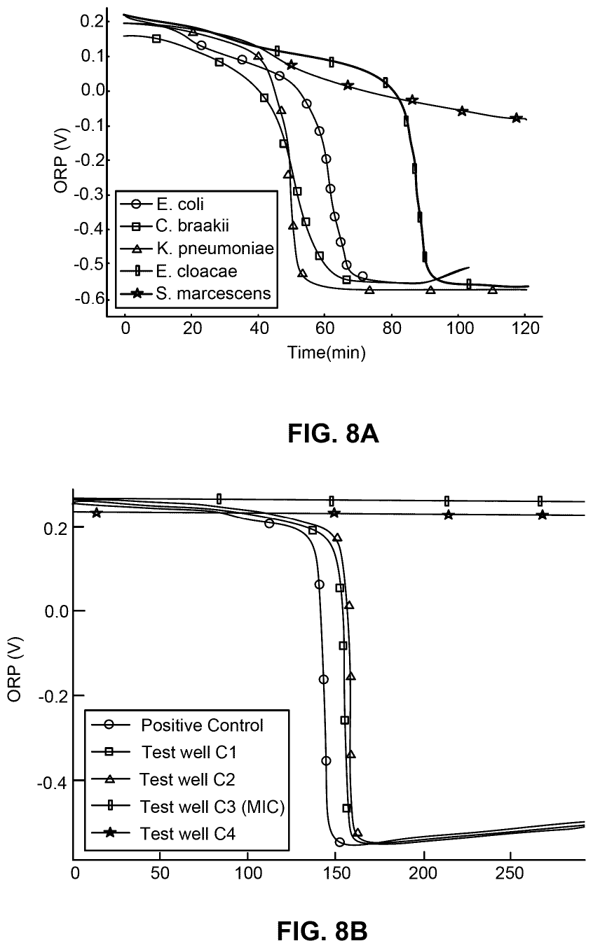

The MCU or one or more other processors within the reader 116 can determine the susceptibility of the bacteria to the antibiotic based on any changes in a solution characteristic (e.g., ORP) of the aliquots within the test wells compared to the control well. For example, when the solution characteristic monitored is ORP, the ORP signals in at least some of the test wells can be compared against the ORP signal from the positive control well (the well devoid of any antibiotic). As will be discussed in more detail in relation to FIG. 8B, the test wells can contain the antibiotic in differing concentrations. Once the ORP signal of a well crosses a predetermined voltage threshold within an allotted time, the one or more processors of the reader 116 can consider the well to be positive for growth. On the other hand, when the ORP signal of a particular well fails to cross the predetermined voltage threshold within the allotted time, the one or more processors of the reader 116 can consider the well to be negative for growth (i.e., bacterial growth has been inhibited).

The MCU can then communicate the data to a single board computer (SBC) within the reader housing 118. The SBC can run the touchscreen graphical user interface (GUI) of the display 120 and certain operating software (including an operating system). The SBC can also interface or communicate with a laboratory information system (LIS).

The SBC can prompt the user when the test is complete via the display 120 and/or via one or more auditory alerts. For example, the display 120 can inform the user that the testing device 110 is ready to be removed from the reader 116 and discarded.

Since the reader 116 comprises multiple reader modules 114 (e.g., up to ten reader modules 114), the reader 116 can read test results from multiple testing devices 110 via random access. In this manner, multiple antibiotics and multiple bacterial species can be assessed at once.

The minimum inhibitory concentration (MIC) for each antibiotic can be reported as a discrete concentration value or as a range based on the initial dilution amounts. The interpretation of the MIC values can be provided according to Clinical & Laboratory Standards Institute (CLSI) or Food and Drug Administration (FDA) guidelines or criteria. The MIC results can be presented via the display 120 and the degree of susceptibility can be shown as susceptible (S), intermediate (I), or resistant®.

Another technical problem faced by the applicants is how to ensure that the reader 116 and the testing device 110 work according to their intended use and performance specifications. One technical solution discovered and/or developed by the applicants is to build in certain internal quality checks prior to and during each testing run. For example, one or more processors of the reader 116 can be programmed to perform the internal quality checks once a user has started a testing run or once the plate tray 400 holding the assembled testing device 110 has been fully inserted or retracted back into the reader housing 118.

In some embodiments, the internal quality check can comprise checking that a baseline voltage of a starting ORP of each of the aliquots of the sample within each of the wells is between about 2600 millivolts (mV) and 2750 mV (e.g., between about 100 mV and 250 mV, plus the 2.5V DC offset). The internal quality check can also comprise checking that a voltage noise of each of the ORP sensors is between about 0 millivolt root-mean-square (mVrms) and 5 mVrms. The internal quality check can further comprise checking that a sensor voltage drift of each of the ORP sensors is between about 0 mV per hour and 40 mV per hour. If the internal quality check reads a consistent value outside of the acceptable range or ranges, an error can be presented via the display 120 asking the user to take an appropriate action (e.g., use a different sensor array lid 112) and end the current run. If the internal quality check is passed, the testing run is allowed to proceed.

FIG. 6 illustrates a side view of a reader module 114 and a close-up view showing a gasket 410 compressed between a sensor array lid 112 of the testing device 110 and the PCBA 406. As previously discussed, the gasket 410 can be adhered or otherwise affixed to an underside of a gasket plate 408. For example, the gasket 410 can be adhered to the underside of the gasket plate 408 by an acrylic adhesive.

In some embodiments, the gasket 410 can be made in part of a semipermeable polymeric material (e.g., silicone rubber) having a Shore A hardness of between about 20 to 55. In certain embodiments, the gasket can be made in part of a semipermeable polymeric material having a Shore A hardness of between about 45 and 50.

As shown in FIG. 6, when the assembled testing device 110 containing aliquots of the sample 102 is inserted into the reader 116 by the plate tray 400 and the tray carrier 402, the gasket plate 408 can be positioned in between the sensor array lid 112 and the PCBA 406 and the gasket 410 can be compressed and create a partial seal around a top of the sensor array lid 112 to control an evaporation rate of the aliquots of the sample 102 and to control a humidity level and a partial pressure of oxygen within the test headspace 700 (see FIG. 7) above the well plate 108.

FIG. 6 also illustrates that the plate tray 400 can comprise a plurality of wheels 600 and the tray carrier 402 can comprise wheel tracks 602 for interfacing with the wheels 600 of the plate tray 400. The wheels 600 of the plate tray 400 can slide or roll along the wheel tracks 602 of the tray carrier 402.

FIG. 7 is a side view illustrating a reader module 114 with a testing device 110 loaded within the reader module 114 and the gasket 410 removed for ease of viewing. Part of the test headspace 700 is shown in FIG. 7 with the gasket 410 removed. The test headspace 700 can refer to the volume of space in between the top of the well plate 108 and the bottom of the PCBA 406.

In some embodiments, the test headspace 700 can have a volume of about 9,012 mm³. Depending on the size of the well plate 108 and the size of the sensor array lid 112, the test headspace 700 can be between about 8000 mm³ and 10,000 mm³.

FIG. 7 also illustrates some of the conductive connectors 500 of the PCBA 406 extending downward from the underside of the PCBA 406.

FIG. 8A is a graph illustrating ORP bacterial growth curves for various bacteria recorded using the system (reader 116 and testing device 110) disclosed herein. ORP measurements for five different species are represented in the graph including E. coli, C. braakii, K. pneumoniae, E. cloacae and S. marcescens. No antibiotics were added to the samples shown in FIG. 8A so the ORP measurements can be considered positive controls for the bacteria indicated.

As the bacteria grows and consumes nutrients present in the growth medium, the redox state of the sample changes. Over time, the ORP signal becomes more negative as the bacterial cell density increases during the growth phase, resulting in a larger concentration of reduced molecules in solution. Since reduced molecules readily give up their electrons, the active electrode immersed in the sample becomes more negative, which results in a decrease in the measured ORP voltage.

FIG. 8B is a graph illustrating ORP measurements for aliquots of a sample containing E. coli in a positive control well devoid of any antibiotic and four test wells comprising differing concentrations of an antibiotic. All graph traces shown in FIG. 8B represent real-time ORP data captured using the reader 116 and testing device 110 disclosed herein. The antibiotic used is part of the monobactam class of antibiotics.

The concentration of the antibiotic increased two-fold for each of the four test wells starting with test well C1, test well C2 (with 2× the concentration of the antibiotic in C1), test well C3 (with 2× the concentration of the antibiotic in C2), and test well C4 (with 2× the concentration of the antibiotic in C3).

The ORP signals for the four test wells were compared to the ORP signal of the positive control well. All ORP signals that exhibited a predetermined voltage change (in this case, crossing the voltage threshold of −0.2 V) within an allotted time window (<200 minutes) were considered “growth.” ORP signals that do not cross the voltage threshold within the allotted time window were considered “no growth.”

As expected, the ORP signals seen in test wells C1 and C2 closely matched the ORP signal of the positive control well and all three ORP signals met the requirements for growth by crossing the −0.2 V threshold within 200 minutes. However, as can be seen in FIG. 8B, the ORP signal for test well C3 did not exhibit this same change in ORP signal magnitude within the allotted time window (<200 minutes), and is, thus, deemed to be “no growth.” The same is also true for test well C4 with the highest concentration of the antibiotic. Therefore, the minimum inhibitory concentration (MIC) is determined to be the antibiotic concentration in test well C3.

Although results with one positive control well and four test wells are depicted and disclosed herein, it is contemplated by this disclosure that the testing device 110 can comprise multiple positive control wells and more than four test wells (anywhere between five test wells and up to 95 test wells). Also, although E. coli is mentioned as the bacteria tested with respect to FIG. 8B, it is contemplated by this disclosure that the testing device 110 can be used for antibiotic susceptibility testing for any of the bacteria listed in this disclosure. Moreover, although a change in the magnitude of the ORP signal is mentioned as the ORP parameter, it is contemplated by this disclosure that the slope of the ORP curve and other features of the ORP curve can also be used to compare the test well(s) against the positive control well.

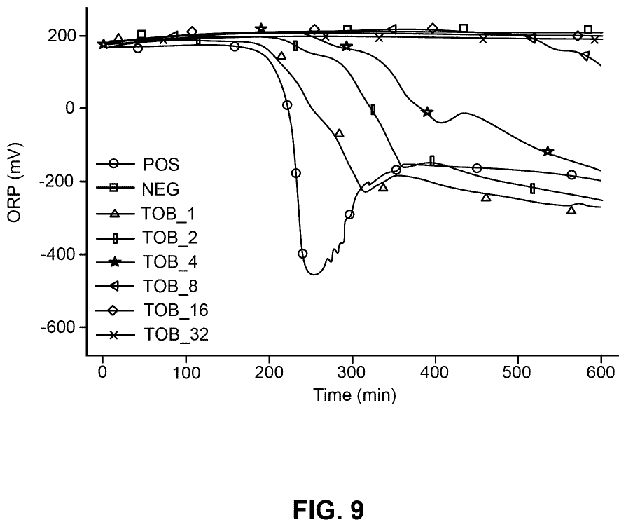

FIG. 9 is a graph illustrating ORP measurements for aliquots of a sample containing Citrobacter spp. in a positive control well (POS) devoid of any antibiotic, a negative control well (NEG) with a known inhibitory concentration of an antibiotic, and six test wells comprising differing concentrations of the antibiotic. All graph traces shown in FIG. 9 represent real-time ORP data captured using the reader 116 and testing device 110 disclosed herein. The antibiotic used was tobramycin (TOB), which is part of the aminoglycoside class of antibiotics.

The concentration of the antibiotic increased two-fold for each of the six test wells beginning with a tobramycin concentration of 1 μg/mL (TOB_1) and ending at a tobramycin concentration of 32 μg/mL (TOB_32). The ORP signals for the six test wells were compared to the ORP signals of the positive and negative control wells. As can be seen in FIG. 9, growth was detected in test wells with tobramycin concentrations of 1 μg/mL, 2 μg/mL, and 4 μg/mL. Therefore, the MIC of the aliquots of the sample containing Citrobacter spp. can be determined to be about 8 μg/mL of tobramycin.

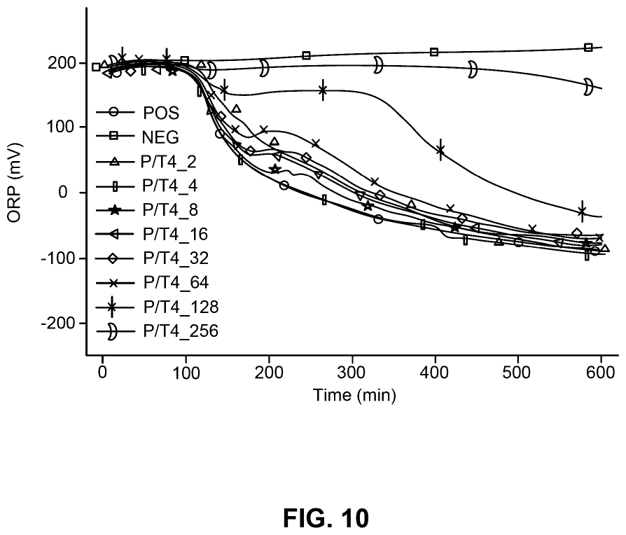

FIG. 10 is a graph illustrating ORP measurements for aliquots of a sample containing the bacteria A. baumannii in a positive control well (POS) devoid of any antibiotic, a negative control well (NEG) with a known inhibitory concentration of an antibiotic, and eight test wells comprising differing concentrations of the antibiotic. All graph traces shown in FIG. 10 represent real-time ORP data captured using the reader 116 and testing device 110 disclosed herein. The antibiotic used was piperacillin tazobactam (P/T4), which is a beta-lactam combination agent.

The concentration of the antibiotic increased two-fold for each of the eight test wells beginning with a piperacillin tazobactam concentration of 2 μg/mL (P/T4_2) and ending at a piperacillin tazobactam concentration of 256 μg/mL (P/T4_256). The ORP signals for the eight test wells were compared to the ORP signals of the positive and negative control wells. As can be seen in FIG. 10, growth was detected in test wells with piperacillin tazobactam concentrations of 2 μg/mL to 128 μg/mL. Therefore, the MIC of the aliquots of the sample containing A. baumannii can be determined to be about 256 μg/mL of piperacillin tazobactam.

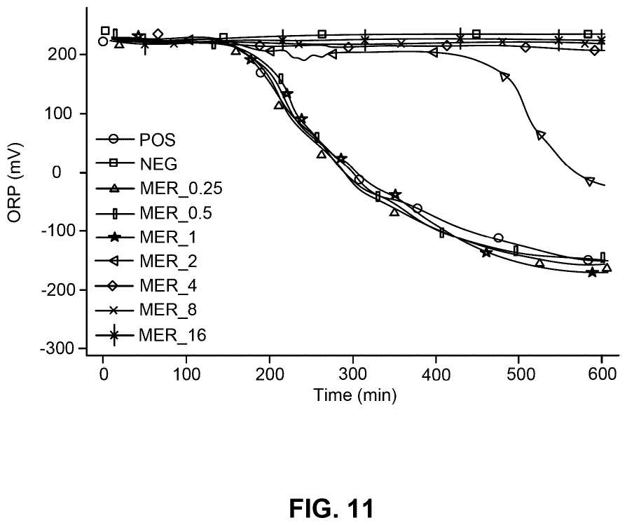

FIG. 11 is a graph illustrating ORP measurements for aliquots of a sample containing the bacteria S. marcescens in a positive control well (POS) devoid of any antibiotic, a negative control well (NEG) with a known inhibitory concentration of an antibiotic, and seven test wells comprising differing concentrations of the antibiotic. All graph traces shown in FIG. 11 represent real-time ORP data captured using the reader 116 and testing device 110 disclosed herein. The antibiotic used was meropenem (MER), which is part of the carbapenem class of antibiotics.

The concentration of the antibiotic increased two-fold for each of the seven test wells beginning with a meropenem concentration of 0.25 μg/mL (MER_0.25) and ending at a meropenem concentration of 16 μg/mL (MER_16). The ORP signals for the seven test wells were compared to the ORP signals of the positive and negative control wells. As can be seen in FIG. 11, growth was detected in test wells with meropenem concentrations of 0.25 μg/mL to 2 μg/mL. Therefore, the MIC of the aliquots of the sample containing S. marcescens can be determined to be about 4 μg/mL of meropenem.

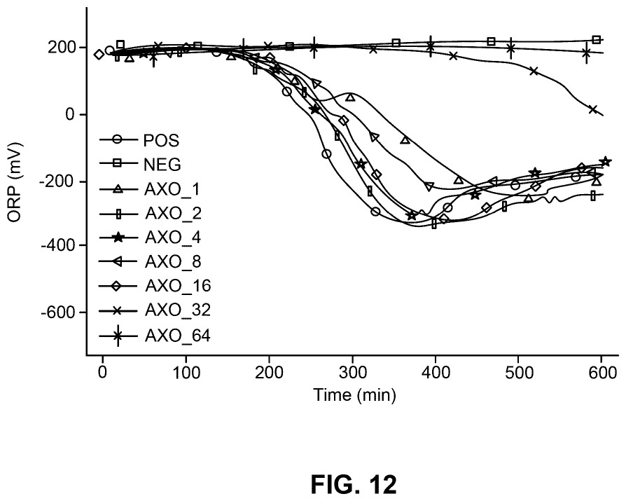

FIG. 12 is a graph illustrating ORP measurements for aliquots of a sample containing K. pneumoniae in a positive control well (POS) devoid of any antibiotic, a negative control well (NEG) with a known inhibitory concentration of an antibiotic, and seven test wells comprising differing concentrations of the antibiotic. All graph traces shown in FIG. 12 represent real-time ORP data captured using the reader 116 and testing device 110 disclosed herein. The antibiotic used is ceftriaxone (AXO), which is part of the cephalosporin class of antibiotics.

The concentration of the antibiotic increased two-fold for each of the seven test wells beginning with a ceftriaxone concentration of 1.0 μg/mL (AXO_1) and ending at a ceftriaxone concentration of 64 μg/mL (AXO_64). The ORP signals for the seven test wells were compared to the ORP signals of the positive and negative control wells. As can be seen in FIG. 12, growth was detected in test wells with ceftriaxone concentrations of 1 μg/mL to 32 μg/mL. Therefore, the MIC of the aliquots of the sample containing K. pneumoniae can be determined to be about 64 μg/mL of ceftriaxone.

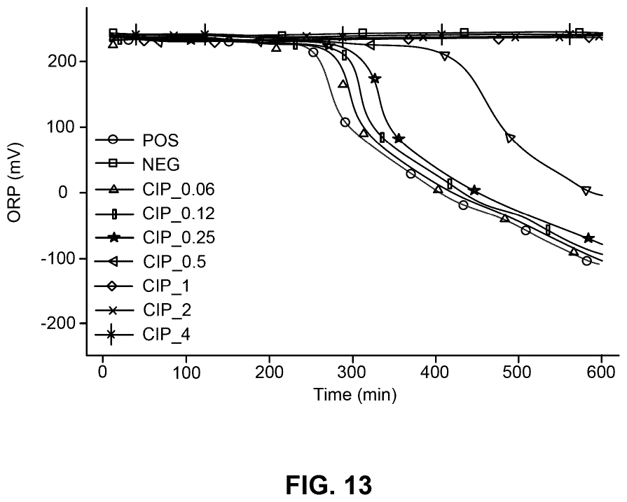

FIG. 13 is a graph illustrating ORP measurements for aliquots of a sample containing P. aeruginosa in a positive control well (POS) devoid of any antibiotic, a negative control well (NEG) with a known inhibitory concentration of an antibiotic, and seven test wells comprising differing concentrations of the antibiotic. All graph traces shown in FIG. 13 represent real-time ORP data captured using the reader 116 and testing device 110 disclosed herein. The antibiotic used is ciprofloxacin (CIP), which is part of the fluoroquinolone class of antibiotics.

The concentration of the antibiotic increased two-fold for each of the seven test wells beginning with a ciprofloxacin concentration of 0.06 μg/mL (CIP_0.06) and ending at a ciprofloxacin concentration of 4 μg/mL (CIP_4). The ORP signals for the seven test wells were compared to the ORP signals of the positive and negative control wells. As can be seen in FIG. 13, growth was detected in test wells with ciprofloxacin concentrations of 0.06 μg/mL to 0.5 μg/mL. Therefore, the MIC of the aliquots of the sample containing P. aeruginosa can be determined to be about 1.0 μg/mL of ciprofloxacin.

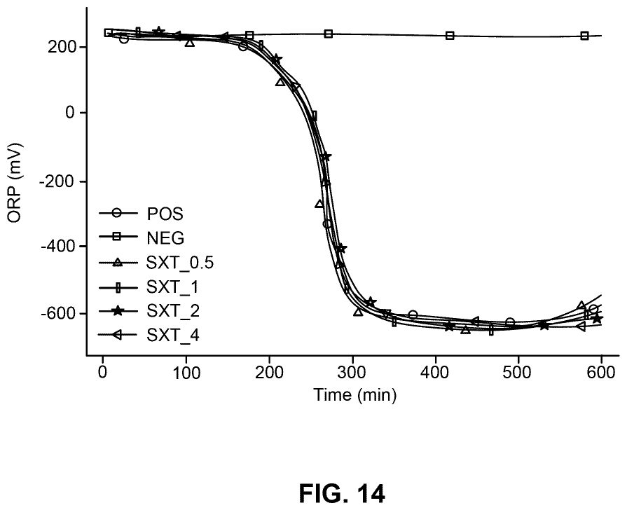

FIG. 14 is a graph illustrating ORP measurements for aliquots of a sample containing Enterobacter cloacae (E. cloacae) in a positive control well (POS) devoid of any antibiotic, a negative control well (NEG) with a known inhibitory concentration of an antibiotic, and four test wells comprising differing concentrations of the antibiotic. All graph traces shown in FIG. 14 represent real-time ORP data captured using the reader 116 and testing device 110 disclosed herein. The antibiotic used is trimethoprim sulfamethoxazole (SXT), which is part of the folate pathway antagonist class of antibiotics.

The concentration of the antibiotic increased two-fold for each of the four test wells beginning with a trimethoprim sulfamethoxazole concentration of 0.5 μg/mL (SXT_0.5) and ending at a trimethoprim sulfamethoxazole concentration of 4 μg/mL (SXT_4). The ORP signals for the four test wells were compared to the ORP signals of the positive and negative control wells. As can be seen in FIG. 14, growth was detected in test wells with trimethoprim sulfamethoxazole concentrations of 0.5 μg/mL to 4 μg/mL. Therefore, the MIC of the aliquots of the sample containing E. cloacae was unable to be determined using the concentrations of trimethoprim sulfamethoxazole shown in FIG. 14.

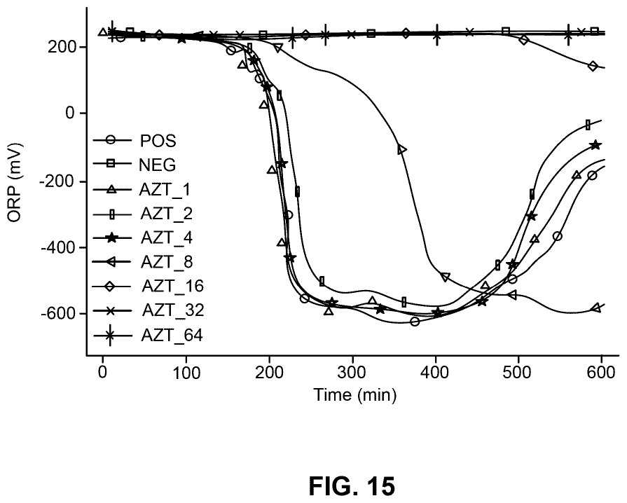

FIG. 15 is a graph illustrating ORP measurements for aliquots of a sample containing E. coli in a positive control well (POS) devoid of any antibiotic, a negative control well (NEG) with a known inhibitory concentration of an antibiotic, and seven test wells comprising differing concentrations of the antibiotic. All graph traces shown in FIG. 15 represent real-time ORP data captured using the reader 116 and testing device 110 disclosed herein. The antibiotic used is aztreonam (AZT), which is part of the monobactam class of antibiotics.

The concentration of the antibiotic increased two-fold for each of the seven test wells beginning with an aztreonam concentration of 1 μg/mL (AZT_1) and ending at an aztreonam concentration of 64 μg/mL (AZT_64). The ORP signals for the seven test wells were compared to the ORP signals of the positive and negative control wells. As can be seen in FIG. 15, growth was detected in test wells with aztreonam concentrations of 1 μg/mL to 8 μg/mL. Therefore, the MIC of the aliquots of the sample containing E. coli can be determined to be about 16 μg/mL of aztreonam.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including,” “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to +0.1%, +1%, +5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. A reader for determining a susceptibility of a bacteria to an antibiotic, comprising:
a plurality of reader modules, wherein each of the reader modules comprises:
a plate tray configured to receive a well plate covered by a sensor array lid, wherein the well plate comprises a plurality of wells configured to contain aliquots of a sample comprising the bacteria, wherein the wells comprise test wells containing the antibiotic and at least one control well devoid of any antibiotic, and wherein the sensor array lid comprises a plurality of sensor units,
a printed circuit board assembly (PCBA) disposed above the plate tray, wherein the PCBA comprises a plurality of conductive connectors configured to extend downward from an underside of the PCBA to contact conductive pads of the sensor array lid, wherein at least part of each of the sensor units is configured to be immersed in an aliquot of the sample within a well of the well plate, and
one or more processors communicatively coupled to the PCBA, wherein the one or more processors are programmed to determine the susceptibility of the bacteria to the antibiotic based on any changes in a solution characteristic of the aliquots within the test wells compared to the control well over time.

2. The reader of claim 1, wherein the conductive connectors are leaf spring connectors.

3. The reader of claim 2, wherein the leaf spring connectors are made in part of a conductive metal or metal alloy.

4. The reader of claim 1, wherein each of the reader modules further comprises a gasket disposed in between the PCBA and the sensor array lid covering the well plate placed on the plate tray, wherein the gasket is configured to create a partial seal around a top of the sensor array lid to control an evaporation rate of the aliquots of the sample and to control a humidity level and a partial pressure of oxygen within a space above the well plate.

5. The reader of claim 4, wherein the gasket is made in part of a semipermeable polymeric material having a Shore A hardness of between about 20 to 55.

6. The reader of claim 4, wherein the gasket serves as a perimeter surrounding portions of the conductive connectors extending downward from the underside of the PCBA.

7. The reader of claim 1, wherein each of the reader modules further comprises an upper heater coupled to the PCBA, and wherein the upper heater is configured to control condensation on the conductive connectors by heating the conductive connectors above a dew point.

8. The reader of claim 7, wherein each of the reader modules further comprises a lower heater coupled to the plate tray, wherein the well plate is configured to be placed above the lower heater, wherein the lower heater is configured to heat the well plate.

9. The reader of claim 8, wherein each of the reader modules further comprises a first temperature sensor configured to monitor a temperature of the lower heater used to heat the well plate.

10. The reader of claim 9, wherein each of the reader modules further comprises a second temperature sensor and a humidity sensor to monitor the temperature and the humidity, respectively, within a space above the well plate.

11. The reader of claim 1, wherein the sensor units of the sensor array lid comprise oxidation-reduction potential (ORP) sensors, wherein the one or more processors are further programmed to perform an internal quality check, wherein the internal quality check comprises checking that a baseline voltage of a starting ORP of each of the aliquots of the sample within each of the wells is between about 2600 millivolts (mV) and 2750 mV.

12. The reader of claim 1, wherein the sensor units of the sensor array lid comprise oxidation-reduction potential (ORP) sensors, wherein the one or more processors are further programmed to perform an internal quality check, wherein the internal quality check comprises checking that a voltage noise of each of the ORP sensors is between about 0 millivolt root-mean-square (mVrms) and 5 mVrms.

13. The reader of claim 1, wherein the sensor units of the sensor array lid comprise oxidation-reduction potential (ORP) sensors, wherein the one or more processors are further programmed to perform an internal quality check, wherein the internal quality check comprises checking that a sensor voltage drift of each of the ORP sensors is between about 0 mV per hour and 40 mV per hour.

14. The reader of claim 1, further comprising a reader housing, wherein each of the reader modules further comprises a tray carrier configured to automatically translate or drive the plate tray at least partially out of the reader housing to receive the well plate covered by the sensor array lid, wherein the conductive connectors are aligned to contact the conductive pads of the sensor array lid after the plate tray loaded with the well plate covered by the sensor array lid is automatically retracted back into the reader housing by the tray carrier.

15. The reader of claim 1, wherein the sensor array lid further comprises a lid cover and a sensor substrate layer coupled to an underside of the lid cover, wherein the lid cover comprises a plurality of openings configured to expose the conductive pads, and wherein the conductive connectors are configured to extend into the openings to contact the conductive pads.

16. A reader for determining a susceptibility of a bacteria to an antibiotic, comprising:
a plurality of reader modules, wherein each of the reader modules comprises:
a plate tray configured to receive a well plate covered by a sensor array lid, wherein the well plate comprises a plurality of wells configured to contain aliquots of a sample comprising the bacteria, wherein the wells comprise test wells containing the antibiotic and at least one control well devoid of any antibiotic, and wherein the sensor array lid comprises a plurality of sensor units,
a printed circuit board assembly (PCBA) disposed above the plate tray and configured to be in electrical contact with the sensor array lid, wherein at least part of each of the sensor units of the sensor array lid is configured to be immersed in an aliquots of the sample within a well of the well plate,
a gasket disposed in between the PCBA and the sensor array lid, wherein the gasket is configured to create a partial seal around a top of the sensor array lid to control an evaporation rate of the aliquots of the sample and a humidity level and a partial pressure of oxygen within a space above the well plate, and
one or more processors communicatively coupled to the PCBA, wherein the one or more processors are programmed to determine the susceptibility of the bacteria to the antibiotic based on any changes in a solution characteristic of the aliquots within the test wells compared to the control well over time.

17.-31. (canceled)

32. A reader for determining a susceptibility of a bacteria to an antibiotic, comprising:
a plurality of reader modules, wherein each of the reader modules comprises:
a plate tray configured to receive a well plate covered by a sensor array lid, wherein the well plate comprises a plurality of wells configured to contain aliquots of a sample comprising the bacteria, wherein the wells comprise test wells containing the antibiotic and at least one control well devoid of any antibiotic, and wherein the sensor array lid comprises a plurality of oxidation-reduction potential (ORP) sensors,
a printed circuit board assembly (PCBA) disposed above the plate tray and configured to be in electrical contact with the sensor array lid, wherein at least part of each of the ORP sensors is configured to be immersed in an aliquot of the sample within a well of the well plate, and
one or more processors communicatively coupled to the PCBA, wherein the one or more processors are programmed to:
check that a baseline voltage of a starting ORP of each of the aliquots of the sample within the wells is between about 2600 millivolts (mV) and 2750 mV, a voltage noise of each of the ORP sensors is between about 0 millivolt root-mean-square (mVrms) and 5 mVrms, and a sensor voltage drift of each of the ORP sensors is between about 0 mV per hour and 40 mV per hour, and
determine the susceptibility of the bacteria to the antibiotic based on any changes in a solution characteristic of the aliquots within the test wells compared to the control well over time.

33.-41. (canceled)

42. One or more non-transitory computer-readable media comprising instructions stored thereon, that when executed by one or more processors, cause the one or more processors to perform operations comprising:
checking that a baseline voltage of a starting oxidation-reduction potential (ORP) of each aliquot of a sample within wells of a well plate is between about 2600 millivolts (mV) and 2750 mV, wherein the sample comprises bacteria, wherein the wells comprise test wells containing an antibiotic and at least one control well devoid of any antibiotic, wherein the well plate is covered by a sensor array lid comprising a plurality of ORP sensors configured to be immersed in the aliquots of the sample within the well plate, wherein the well plate is placed on a plate tray of reader module, wherein the reader module further comprises a printed circuit board assembly (PCBA) disposed above the plate tray and configured to be in electrical contact with the sensor array lid;
checking that a voltage noise of each of the ORP sensors is between about 0 millivolt root-mean-square (mVrms) and 5 mVrms;
checking that a sensor voltage drift of each of the ORP sensors is between about 0 mV per hour and 40 mV per hour; and
determining a susceptibility of the bacteria to the antibiotic based on any changes in a solution characteristic of the aliquots within the test wells compared to the control well over time.

43. A method of determining a susceptibility of a bacteria to an antibiotic, comprising:
placing a well plate covered by a sensor array lid on a plate tray of a reader module of a reader, wherein the well plate comprises a plurality of wells configured to contain aliquots of a sample comprising the bacteria, and wherein the wells comprise test wells containing the antibiotic and at least one control well devoid of any antibiotic, and wherein sensor units of the sensor array lid are configured to be immersed in the aliquots of the sample within the well plate;
pushing the plate tray into the reader or causing the plate tray to be retracted into the reader, wherein a plurality of conductive connectors of a printed circuit board assembly (PCBA) disposed above the plate tray is placed in electrical contact with conductive pads of the sensor array lid after the plate tray is fully pushed or retracted into the reader, wherein the conductive connectors extend downward from an underside of the PCBA to contact the conductive pads of the sensor array lid; and
determining, using one or more processors communicatively coupled to the PCBA, the susceptibility of the bacteria to the antibiotic based on any changes in a solution characteristic of the aliquots within the test wells compared to the control well over time.

44.-51. (canceled)
